(19) United States
(12) Patent Application Publication
(10) Pub. No.: US 2009/0041801 A1
(43) Pub. Date: Feb. 12, 2009

(54) Metal Chelator

(76) Inventor: George J. Georgiou, Larnaca (CY)

Correspondence Address: HOWSON AND HOWSON, SUITE 210, 501 OFFICE CENTER DRIVE, FT WASHINGTON, PA, 19034, US

(21) Appl. No.: 11/834,431

(22) Filed: Aug. 6, 2007

(51) Int. Cl. A61K 36/05; A61P 39/04

(52) U.S. Cl. 424/195.17

(57) ABSTRACT

A natural substance composition for the elimination of toxic metals from the body comprising a homeopathic detox factor, a nucleotide-peptide complex and coriander sativum.

### BACKGROUND OF THE INVENTION

The present invention relates to a natural substance composition that is a heavy metal chelator, and more especially to a natural substance composition that chelates Lead, Arsenic, Cadmium and Antimony.

Foundry workers are exposed to a number of toxic heavy metals over the course of their normal working lives. Exposure to these metals causes concern as their long term effects and accumulative properties, both for workers and for the companies employing them.

It is therefore desirable to have a system by which the toxic levels of these metals in the bodies of the foundry workers, and other people with regular exposure, can be reduced to reduce the possibility of any undesirable side effects from the toxicity of the metals.

A natural substance composition that is a heavy metal chelator on the market is NDF (Nano Detox Factor) as well as Metal-Free which claim to show good results for aiding the removal of these metals from the body, but the cost per unit is too high ($120-$150) and is economically unsustainable for companies with large workforces exposed to these toxic substances. Moreover, no double blind, placebo controlled studies have been conducted on these substances to date; so the data presented to support their efficiency is scant and incomplete.

There are a number of drug-type substances on the market that are traditionally used for heavy metal chelation, for example EDTA, DMPS, DMSA, but these are also expensive and have many unwanted side-effects, particularly when used with workers who are occupationally exposed to metals such as foundry workers and miners.

There are no other economically viable and readily available natural substances currently on the market know to be effective for all these metals simultaneously.

### SUMMARY OF THE INVENTION

The aim of the present invention is to provide a cost effective natural source composition heavy metal chelator for use in enabling the removal of a range of heavy metals from the human body without any undue side effects.

Accordingly, the present invention is directed to a natural substance composition for the elimination of toxic metals from the body comprising a homeopathic detox factor, a nucleotide-peptide complex and coriander sativum.

Preferably, the homeopathic detox factor contains cell wall decimated Chlorella, herb extracts and bacteriocin components of probiotics.

Preferably, the nucleotide-peptide complex is Chlorella Growth Factor.

Advantageously, the composition contains between 10% and 50% Homeopathic Nano Detox Factor.

Preferably, the composition contains 20% Homeopathic Nano Detox Factor.

Advantageously, the composition contains 30% Homeopathic Nano Detox Factor.

Preferably, the composition contains 50% nucleotide-peptide complex.

Advantageously, the composition contains 40% nucleotide-peptide complex.

Preferably, the composition contains between 20% and 40% of Cilantro.

Advantageously, the composition contains 30% of Cilantro.

### BRIEF DESCRIPTION OF THE DRAWINGS

FIG. 1 shows the levels of Pb in Urine in the Control Group;

FIG. 2 shows the levels of Pb in Urine in Group 3 (60 drops×3 daily) using Homeopathic chlorella;

FIG. 3 shows the levels of Pb in Urine in Group 5 (60 drops×3 daily) using Homeopathic chlorella, cilantrin and CGF;

FIG. 4 shows the levels of Pb in Urine in Group 6 (40 drops×3 daily) using Homeopathic chlorella, CGF and Cilantro;

FIG. 5 shows the levels of Pb in Urine in Group 6 (60 drops×3 daily) using Homeopathic chlorella, CGF and Cilantro;

FIG. 6 shows the level of Pb in Faeces in the Control Group (60 drops×3 daily) using placebo;

FIG. 7 shows levels of Pb in Faeces in Group 3 (60 drops×3 daily) using Homeopathic chlorella;

FIG. 8 shows the levels of Pb in Faeces in Group 4 (40 drops×3 daily) using Homeopathic chlorella, CGF and cilantrin;

FIG. 9 shows level of Pb in Faeces in Group 4 (50 drops×3 daily) using Homeopathic chlorella, CGF and cilantrin;

FIG. 10 shows levels of Pb in Faeces in Group 5 (40 drops×3 daily) using Homeopathic chlorella, CGF and cilantrin;

FIG. 11 shows levels of Pb in faeces in Group 5 (50 drops×3 daily) using Homeopathic chlorella, CGF and Cilantro;

FIG. 12 shows levels of Pb in Faeces in Group 7 (40 drops×3 daily) using Homeopathic chlorella, CGF and Cilantro;

FIG. 13 shows levels of Pb in Faeces in Group 7 (50 drops×3 daily) using Homeopathic chlorella, CGF and Cilantro;

FIG. 14 shows levels of Arsenic in Urine in the Control Group (60 drops×3 daily);

FIG. 15 shows levels of As in Urine in Group 3 (60 drops×3 daily) using Homeopathic chlorella;

FIG. 16 shows levels of As in Urine in Group 4 (40 drops×3 daily) using Homeopathic chlorella, CGF and Cilantro;

FIG. 17 shows levels in As in Urine in Group 4 (50 drops×3 daily) using Homeopathic chlorella, CGF and Cilantro;

FIG. 18 shows levels of As in Urine in Group 4 (60 drops×3 daily) using Homeopathic chlorella, CGF and Cilantro;

FIG. 19 shows levels of As in Urine in Group 5 (40 drops×3 daily) using Homeopathic chlorella, CGF and Cilantro;

FIG. 20 shows levels of As in Urine in Group 5 (50 drops×3 daily) using Homeopathic chlorella, CGF and Cilantro;

FIG. 21 shows levels of As in Urine in Group 5 (60 drops×3 daily) using Homeopathic chlorella, CGF and Cilantro;

FIG. 22 shows levels of As in Urine in Group 6 (40 drops×3 daily) using Homeopathic chlorella, CGF and Cilantro;

FIG. 23 shows levels of As in Urine in Group 6 (50 drops×3 daily) using Homeopathic chlorella, CGF and Cilantro;

FIG. 24 shows levels of As in Urine in Group 6 (60 drops×3 daily) using Homeopathic chlorella, CGF and Cilantro;

FIG. 25 shows levels of As in Faeces in the Control Group (60 drops×3 daily);

FIG. 26 shows levels of As in Faeces in Group 4 (40 drops×3 daily) using Homeopathic chlorella, CGF and Cilantro;

FIG. 27 shows levels of As in Faeces in Group 4 (50 drops×3 daily) using Homeopathic chlorella, CGF and Cilantro;

FIG. 28 shows levels of As in Faeces in Group 5 (50 drops×3 daily) using Homeopathic chlorella, CGF and Cilantro;

FIG. 29 shows levels of As in Faeces in Group 6 (40 drops×3 daily) using Homeopathic chlorella, CGF and Cilantro;

FIG. 30 shows levels of As in Faeces in Group 6 (50 drops×3 daily) using Homeopathic chlorella, CGF and Cilantro;

FIG. 31 shows levels of As in Faeces in Group 7 (40 drops×3 daily) using Homeopathic chlorella, CGF and Cilantro;

FIG. 32 shows levels of As in Faeces in Group 7 (50 drops×3 daily) using Homeopathic chlorella, CGF and Cilantro;

FIG. 33 shows levels in As in Faeces in Group 7 (60 drops×3 daily) using Homeopathic chlorella, CGF and Cilantro;

FIG. 34 shows levels of Cd in Urine in the Control Group;

FIG. 35 shows levels of Cd in Urine in Group 3 (50 drops×3 daily) using Homeopathic chlorella;

FIG. 36 shows levels of Cd in Urine in Group 5 (60 drops×3 daily) using Homeopathic chlorella, CGF and Cilantro;

FIG. 37 shows levels of Cd in Urine in Group 6 (60 drops×3 daily) using Homeopathic chlorella, CGF and Cilantro;

FIG. 38 shows levels of Cd in Faeces in the Control Group;

FIG. 39 shows levels of Cd in Faeces in the Control Group;

FIG. 40 shows levels of Cd in Faeces in Group 4 (40 drops×3 daily) using Homeopathic chlorella, CGF and Cilantro;

FIG. 41 shows levels of Cd in Faeces in Group 5 (40 drops×3 daily) using Homeopathic chlorella, CGF and Cilantro;

FIG. 42 shows levels of Cd in Faeces in Group 6 (60 drops×3 daily) using Homeopathic chlorella, CGF and Cilantro;

FIG. 43 shows levels of Cd in Faeces in Group 7 (40 drops×3 daily) using Homeopathic chlorella, CGF and Cilantro;

FIG. 44 shows levels of Sb in Urine in the Control Group;

FIG. 45 shows levels of Sb in Urine (60 drops×3 daily) using Homeopathic chlorella;

FIG. 46 shows levels of Sb in Urine in Group 4 (50 drops×3 daily) using Homeopathic chlorella, CGF and Cilantro;

FIG. 47 shows levels of Sb in Urine in Group 5 (40 drops×3 daily) using Homeopathic chlorella, CGF and Cilantro;

FIG. 48 shows levels of Sb in Urine in Group 5 (50 drops×3 daily) using Homeopathic chlorella, CGF and Cilantro;

FIG. 49 shows levels of Sb in Urine in Group 6 (50 drops×3 daily) using Homeopathic chlorella, CGF and Cilantro;

FIG. 50 shows levels of Sb in Urine in Group 6 (60 drops×3 daily) using Homeopathic chlorella, CGF and Cilantro;

FIG. 51 shows levels of Sb in Urine in Group 7 (40 drops×3 daily) using Homeopathic chlorella, CGF and Cilantro;

FIG. 52 shows levels of Sb in Urine in Group 7 (50 drops×3 daily) using Homeopathic chlorella, CGF and Cilantro;

FIG. 53 shows levels of Sb in Faeces in the Control Group (60 drops×3 daily);

FIG. 54 shows levels of Sb in Faeces in Group 3 (60 drops×3 daily) using Homeopathic chlorella;

FIG. 55 shows levels of Sb in Faeces in Group 5 (60 drops×3 daily) using Homeopathic chlorella, CGF and Cilantro;

FIG. 56 shows the percentage changes in levels of Antimony in Urine over a 24 hour period in Treatment Group 2 using Cilantrin Tincture;

FIG. 57 shows the percentage changes in levels of Antimony in Faeces over a 48 hour period in Treatment Group 2 using Cilantrin Tincture;

FIG. 58 shows the percentage changes in levels in Arsenic in Faeces over a 48 hour period in Treatment Group 2 using Cilantrin Tincture;

FIG. 59 shows percentage changes in levels of Lead in Faeces over a 24 hour period in Treatment Group 2 using Cilantrin Tincture;

FIG. 60 shows percentage changes in levels of Lead in Faeces over a 48 hour period in Treatment Group 2 using Cilantrin Tincture;

FIG. 61 shows percentage changes in levels of Lead in Urine over a 24 hour period in Treatment Group 2 using Cilantrin Tincture;

FIG. 62 shows percentage changes in levels of Cadmium in Urine over a 24 hour period in Treatment Group 2 using Cilantrin Tincture; and

FIG. 63 shows percentage changes in levels of Cadmium in Faeces over a 48 hour period in Treatment Group 2 using Cilantrin Tincture.

### DETAILED DESCRIPTION OF THE INVENTION

Research was conducted to determine in more detail a heavy metal chelator that would be most effective at chelating four common toxic metals present in the workforce at a metal foundry, namely lead, arsenic, cadmium and antimony.

The research included seven different groups taking different quantities of natural substances compared to a control group. There were approximately 84 participants in total with a maximum of 12 subjects in each group.

Group 1 was the Placebo or Control Group who were given Water and cogniac;
Group 2 was the Cilantro Group consisting of Coriander sativum only;

### Group 3 was the Homeopathic Chlorella Group; and

### Groups 4-7 are the Homeopathic Chlorella+Chlorella Growth Factor (CGF)+Cilantro

Groups each with a different amount of the components present in the compound.

Each of the groups 4 to 7 were further divided into three subgroups consisting of four people in each—each of these subgroups were given the same combination but in different combinations and different quantities: 40 drops×3 times a day, 50 drops×3 times a day and 60 drops×3 times a day. The groups were divided up as follows:

Group 4—Homeopathic chlorella (50%)+CGF (20%)+Cilantro (30%);
Group 5—Homeopathic chlorella (40%)+CGF (30%)+Cilantro (30%);
Group 6—Homeopathic chlorella (30%)+CGF (40%)+Cilantro (30%); and
Group 7—Homeopathic chlorella (20%)+CGF (50%)+Cilantro (30%).

The groups were invited to the conference room over the weekend and the research explained to them in detail. As participation was on a voluntary basis, each person signed a Consent Form, which had clear criteria for participation in the research. There were 6-7 people that decided not to participate in the research and they were allowed to leave without further participation.

The research design was a double blind, placebo-controlled study neither did participants, researchers nor clinical chemists doing the spectrometry analysis know which treatment protocol each participant belonged to. The coding was stored in the coordinator's safe until it was time to interpret the statistical data.

Each participant had to take a baseline sample of urine (24-hour collection) and faeces before beginning to take any compositions. These were returned to the research team and carefully recorded in preparation for X-ray fluorescence spectrometry using the foundry's in-house instrument. Both urine and faeces were taken, as some toxic metals prefer the urinary route of exit while others the biliary route, being eliminated through the faeces.

None of the participants continued working in the foundry as it was important for them not to continue being exposed to the heavy metals during the treatment trials.

After the baseline samples were collected, each participant was given their specific detoxification composition, which they began to take three times per day (see Treatment Protocols above). Urine samples were collected for a full 24 hours during the treatment protocols and faeces for 48 hours due to the slower transit time. These samples were again returned to the research team, coded and sent to the laboratory for analysis. In addition, all participants took Chlorella tablets—500 mg×3 times a day in order to prevent the reabsorption of metals from the gut due to the high levels of toxic metals that these people have—this was an added precaution to prevent any adverse reactions.

All analyses for heavy metals were run on the foundry's own in-house Spectra X-ray Fluorescence spectrometer. All pre and post urine and faeces samples were also prepared in the in-house laboratory under the supervision of the head chemist who had many years experience working in this field.

### Results

Summaries of results are presented below. Only the protocols that were effective at detoxifying the four metals tested, i.e. lead, antimony, arsenic and cadmium (these were the most common metals found in ALL the participants, and the major by-products at the foundry) are presented here.

The results will be presented for each metal in urine and then in faeces. The results are expressed as percentage increases (+) or percentage decreases (−) of heavy metals in urine or faeces. If there is a percentage increase in the post-sample this is an indication that the body is eliminating these metals from the body—an indication that the chelating agent is working successfully. A percentage decrease is an indication that no metals are being eliminated from the body, as in the case when using the placebo. The criteria for choosing a successful composition was that the Total Mean (%) in the group of people being tested had to be positive—the higher the positive number, the more metals were being eliminated.

### Lead in Urine

### Placebo or Control Group

The changes in lead in urine pre- and post testing using the control group who were NOT taking any active substances apart from mineral water with a few drops of cognac added to mask the flavour. Ideally, there should have been no difference in the percentage of metals present in the urine pre and post testing, but there are always individual biochemical differences, which give somewhat different results for each person.

The graph below shows the percentage increase or decrease of lead in urine for the control group. There appear to be some minor fluctuations in the levels with a tendency for the level of lead to decrease when comparing the pre and post samples—there was a mean decrease of lead in urine in the post-test sample of −16.95% showing that the placebo was not chelating lead from the body.

The variations are probably related to the individual biochemical differences and the efficiency of the detoxification systems such as the liver, kidneys, skin, lymphatics, etc. These fluctuations must obviously be taken into account when comparing the treatment protocols. At least one subject continued to work in the foundry for an additional day after the treatment trials began—this additional exposure may have kept the levels of metals high in the body.

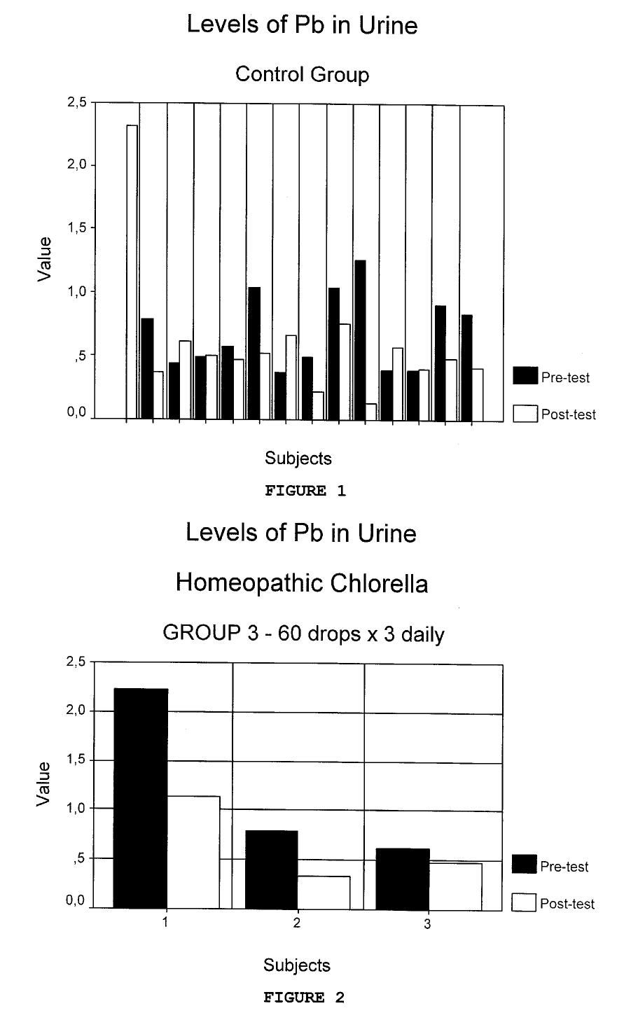

FIG. 1 shows the levels of Pb in Urine in the Control Group.

Case Summaries—the means represent the amount of metals increasing (+) or decreasing (−) in the post sample compared to the pre-sample.

### Placebo Group

60 drops×3 times a day

| SUBJECTS | MEANS (%) |
|---|---|
| 1 | 2.04 |
| 2 | −53.16 |
| 3 | −50.00 |
| 4 | −47.25 |
| 5 | 2.56 |
| 6 | −26.92 |
| 7 | 81.08 |
| 8 | −51.19 |
| 9 | 46.15 |
| 10 | −55.10 |
| 11 | −89.68 |
| 12 | −17.54 |
| 13 | 38.64 |
| TOTAL MEANS (%) | −16.95 |

The Placebo group at the quantity of 60 drops×3 times a day did not show any chelating ability in the post-treatment group, i.e. the total mean is negative at −16.95%.

The Homeopathic chlorella was tried by itself to see whether it was effective at detoxifying Lead and other metals. Without the other synergistic ingredients, there was no increase in metals when comparing the pre and post-tests. Overall there was a 42.95% DECREASE in lead, indicating that the Homeopathic chlorella by itself was not chelating this metal. The amount used was the maximal of 60 drops×3 times a day and the trial was run over 24 hours.

FIG. 2 shows the levels of Pb in Urine in Group 3 (60 drops×3 daily) using Homeopathic chlorella.

Case Summaries—the means represent the amount of metals increasing (+) or decreasing (−) in the post sample compared to the pre-sample.

### Homeopathic Chlorella Group

60 drops×3 times a day

| SUBJECTS | MEAN (%) |
|---|---|
| 1 | −56.96 |
| 2 | −49.33 |
| 3 | −22.58 |
| MEAN TOTAL (%) | −42.95 |

### Homeopathic Chlorella+CGF+Cilantro

The protocol that worked most effectively for eliminating LEAD in URINE, based on this data, is the Homeopathic Chlorella+CGF+Cilantro. There were various quantities that were effective, namely 60 drops×3 times a day, 40 drops×3 times a day. The graph below (Group 5) shows the 77% mean increase in lead in the post-treatment urine samples (Group 5=Homeopathic chlorella (40%)+CGF (30%)+Cilantro (30%).

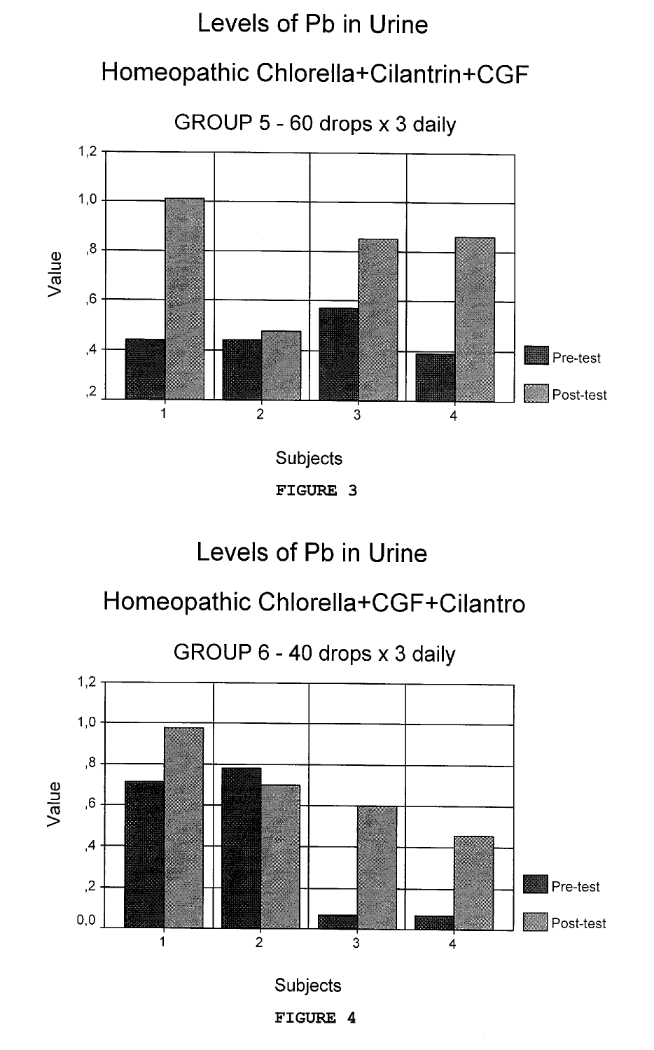

FIG. 3 shows the levels of Pb in Urine in Group 5 (60 drops×3 daily) using Homeopathic chlorella, cilantrin and CGF.

Case Summaries—the means represent the amount of metals increasing (+) or decreasing (−) in the post sample compared to the pre-sample.

Homeopathic chlorella (40%)+CGF (30%)+Cilantro (30%) Group
60 drops×3 times a day

| SUBJECTS | MEANS (%) |
|---|---|
| 1 | +129.5% |
| 2 | +9.09% |
| 3 | +49.1% |
| 4 | +120.5% |
| Total for all subjects | +77.0% |

The graph below (Group 6) shows the 335.5% mean increase in lead in the post-treatment urine sample for a quantity of 40 drops×3 times a day (Group 6 Homeopathic chlorella (30%)+CGF (40%)+Cilantro (30%).

FIG. 4 shows the levels of Pb in Urine in Group 6 (40 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%) Group 40 drops×3 times a day

| SUBJECTS | MEANS (%) |
|---|---|
| 1 | +557.14 |
| 2 | +38.03 |
| 3 | −10.26 |
| 4 | +757.14 |
| TOTAL MEANS | +335.51 |

The graph below (Group 6) shows the 466.4% mean increase in lead in the post-treatment urine sample for a quantity of 60 drops×3 times a day (Group 6=Homeopathic chlorella (30%)+CGF (40%)+Cilantro (30%). It appears that there is a quantity-dependent relationship with this natural compound—60 drops×3 times a day seem to be more effective at eliminating lead in urine compared to 40 drops×3 times a day (446.4% increase compared to 335.5%).

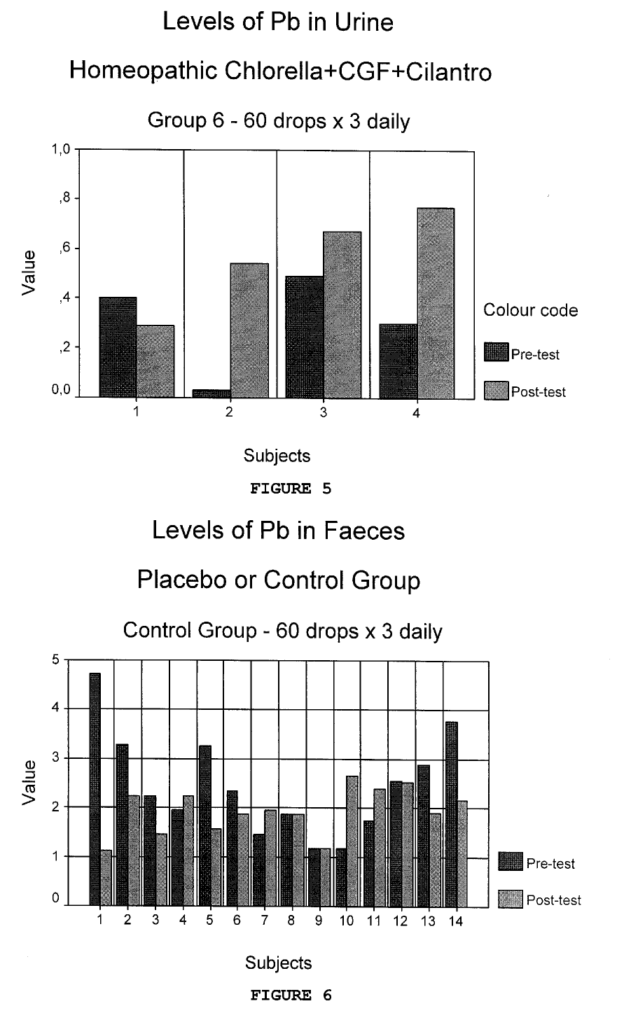

FIG. 5 shows the levels of Pb in Urine in Group 6 (60 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (30%)+CGF (40%)+Cilantro (30%) Group
60 drops×3 times a day

| SUBJECTS | MEANS (%) |
|---|---|
| 1 | −27.5 |
| 2 | 1700.00 |
| 3 | 36.73 |
| 4 | 156.67 |
| TOTAL MEANS | 466.47 |

### Lead Eliminated in Faeces

### Placebo Group

The placebo or control group data to determine the norms for detoxification of lead in faeces. The mean level of lead for this control group was −6.0, meaning that the placebo group did not eliminate any metals in the post-provocation test compared to the pre-provocation test. There were some individual differences due to biochemical individuality, efficiency of detoxification organs, etc., but overall the placebo was not chelating metals from the body. The quantity was 60 drops×3 times a day.

FIG. 6 shows the level of Pb in Faeces in the Control Group (60 drops×3 daily) using placebo.

Case Summaries—the means represent the amount of metals increasing (+) or decreasing (−) in the post sample compared to the pre-sample.

### Placebo or Control Group

60 drops×3 times a day

| SUBJECTS | MEANS (%) |
|---|---|
| 1 | 14.29 |
| 2 | −75.80 |
| 3 | −31.71 |
| 4 | −19.66 |
| 5 | −34.03 |
| 6 | −1.17 |
| 7 | .00 |
| 8 | 31.76 |
| 9 | −42.55 |
| 10 | 36.36 |
| 11 | .00 |
| 12 | 124.58 |
| 13 | −51.53 |
| 14 | −34.72 |
| TOTAL MEAN (%) | −6.0136 |

### Homeopathic Chlorella Protocol

When using Homeopathic chlorella alone there was no chelation of metals demonstrated. The Case Summaries below show that there was a −42.24% decrease of lead in the faeces of the post-sample compared to that of the pre-sample.

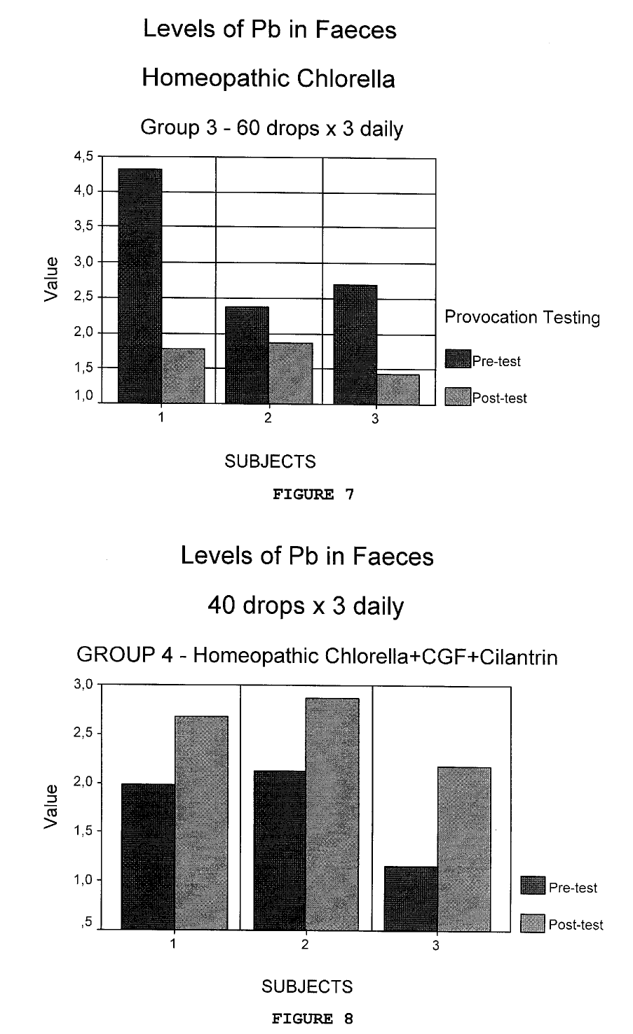

FIG. 7 shows levels of Pb in Faeces in Group 3 (60 drops×3 daily) using Homeopathic chlorella.

Case Summaries—the means represent the amount of metals increasing (+) or decreasing (−) in the post sample compared to the pre-sample.

### Homeopathic Chlorella Group

60 drops×3 times a day

| SUBJECTS | MEANS (%) |
|---|---|
| 1 | −21.10 |
| 2 | −58.80 |
| 3 | −46.84 |
| MEAN TOTALS (%) | −42.24 |

### Effective Chelating Protocols

### Homeopathic Chlorella+Chlorella Growth Factor (CGF)+Cilantro Protocol

The compound that was most effective in the chelation of lead in faeces was the Homeopathic chlorella+CGF+Cilantro. There were a number of compounds that were effective, all with slightly different percentages of the varying natural elements that made up the constituents of the compound.

In the graph below we can see that the Homeopathic chlorella (50%)+CGF (20%)+Cilantro (30%) at a quantity of 40 drops×3 times a day was eliminating 52.67% (see Case Summaries) lead in the faeces in the post-test compared to the pre-test.

FIG. 8 shows the levels of Pb in Faeces in Group 4 (40 drops×3 daily) using Homeopathic chlorella, CGF and cilantrin.

Case Summaries—the means represent the amount of metals increasing (+) or decreasing (−) in the post sample compared to the pre-sample.

Homeopathic chlorella (50%)+CGF (20%)+Cilantro (30%)
40 drops×3 times a day

| SUBJECTS | MEANS (%) |
|---|---|
| 1 | 87.93 |
| 2 | 35.35 |
| 3 | 34.74 |
| MEAN TOTALS (%) | 52.67 |

In the graph below we can see that the Homeopathic chlorella (50%)+CGF (20%)+Cilantro (30%) at a quantity of 50 drops×3 times a day was chelating 142.1% lead in the faeces in the post-test compared to the pre-test. Again we see a quantity-dependent relationship with 50 drops×3 times a day eliminating considerably more than the 40 drops×3 times a day.

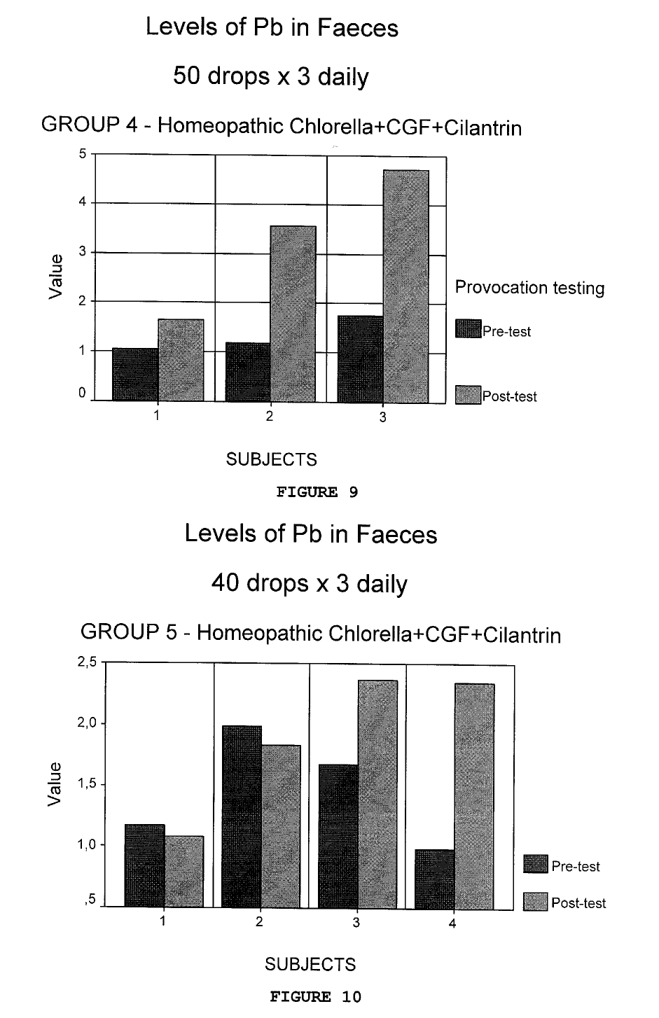

FIG. 9 shows level of Pb in Faeces in Group 4 (50 drops×3 daily) using Homeopathic chlorella, CGF and cilantrin.

Case Summaries—the means represent the amount of metals increasing (+) or decreasing (−) in the post sample compared to the pre-sample.

Group 4—Homeopathic chlorella (50%)+CGF (20%)+Cilantro (30%)
50 drops×3 times a day

| SUBJECTS | MEANS (%) |
|---|---|
| 1 | 56.60 |
| 2 | 168.18 |
| 3 | 201.69 |
| MEAN TOTAL (%) | 142.16 |

### Other Compounds in Varying Concentrations

There were other compounds made from Homeopathic chlorella (40%)+CGF (30%)+Cilantro (30%) which contained slightly less Homeopathic chlorella (40% instead of the previous 50%) and slightly more CGF (30% instead of 20%) while the amount of Cilantro stayed the same. The graph below shows that this compound was eliminating 41.39% (see Case Summaries below) of lead in the post-test faeces compared to the pre-test.

FIG. 10 shows levels of Pb in Faeces in Group 5 (40 drops×3 daily) using Homeopathic chlorella, CGF and cilantrin.

Case Summaries—the means represent the amount of metals increasing (+) or decreasing (−) in the post sample compared to the pre-sample.

Homeopathic chlorella (40%)+CGF (30%)+Cilantro (30%)
40 drops×3 times a day

| SUBJECTS | MEANS (%) |
|---|---|
| 1 | −7.69 |
| 2 | −7.58 |
| 3 | 41.07 |
| 4 | 139.80 |
| MEAN TOTAL (%) | 41.39 |

When the same compound Homeopathic chlorella (40%)+CGF (30%)+Cilantro (30%) was tested using a larger quantity of 50 drops×3 times a day, a 23% increase in the elimination of lead in the faeces was seen on post-testing, compared to pre-testing. This is somewhat less than the previous quantity of 40 drops×3 times a day, making the lower quantity more effective at eliminating lead in faeces. There was an even lower elimination when the quantity was stepped up to 60 drops×3 times a day—7.52% increase.

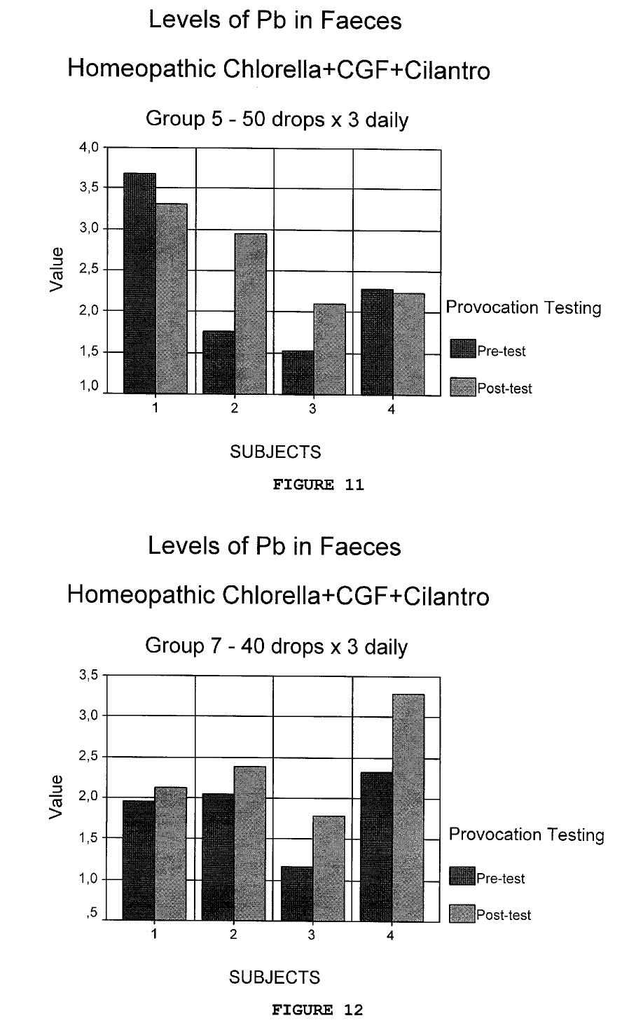

FIG. 11 shows levels of Pb in faeces in Group 5 (50 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (40%)+CGF (30%)+Cilantro (30%)
50 drops×3 times a day

| SUBJECTS | MEANS (%) |
|---|---|
| 1 | −10.08 |
| 2 | 67.61 |
| 3 | 37.25 |
| 4 | −2.62 |
| MEAN TOTALS (%) | 23.04 |

60 drops×3 times a day

| SUBJECTS | MEANS (%) |
|---|---|
| 1 | −18.45 |
| 2 | −40.98 |
| 3 | 59.13 |
| 4 | 30.41 |
| MEAN TOTALS (%) | 7.52 |

The compound Homeopathic chlorella (30%)+CGF (40%)+Cilantro (30%) had marginal success at eliminating lead in the urine, so is not included in this analysis.

The compound Homeopathic chlorella (20%)+CGF (50%)+Cilantro (30%) at a quantity of 40 drops×3 times a day showed a 30.18% increase in the elimination of lead in the faeces in the post-test.

FIG. 12 shows levels of Pb in Faeces in Group 7 (40 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (20%)+CGF (50%)+Cilantro (30%)
40 drops×3 times a day

| SUBJECTS | MEAN (%) |
|---|---|
| 1 | 9.23 |
| 2 | 16.67 |
| 3 | 53.45 |
| 4 | 41.38 |
| MEAN TOTALS (%) | 30.1813 |

The compound Homeopathic chlorella (20%)+CGF (50%)+Cilantro (30%) at a quantity of 50 drops×3 times a day showed a 247.19% increase in the elimination of lead in the faeces in the post-test. The higher quantity of 50 drops×3 times a day showed a significant increase in the elimination of metals and appears to be better than the 40 drops×3 times a day quantity of the same compound.

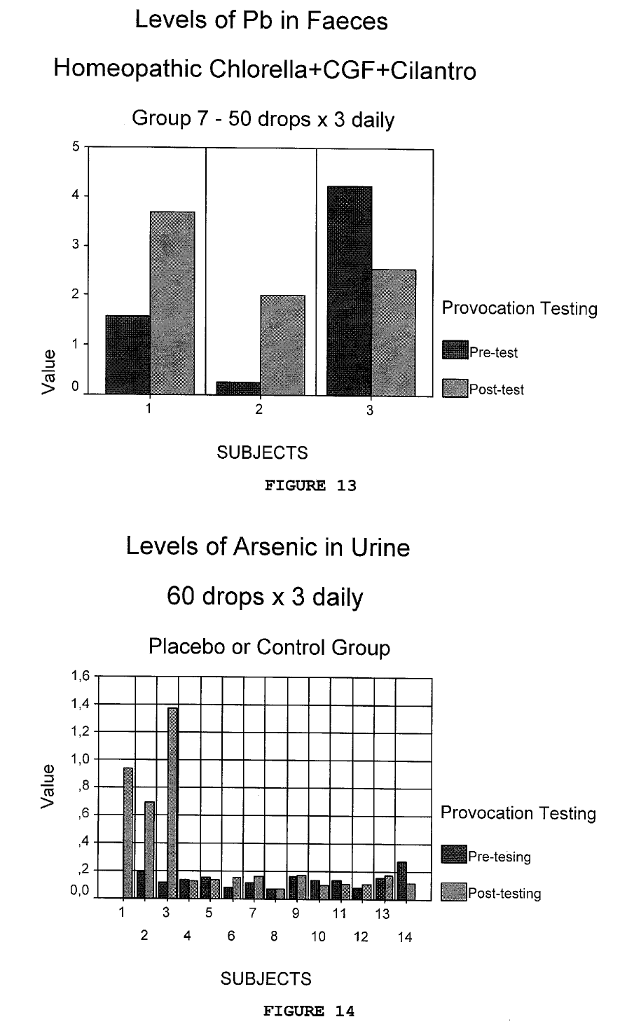

FIG. 13 shows levels of Pb in Faeces in Group 7 (50 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (20%)+CGF (50%)+Cilantro (30%)
50 drops×3 times a day

| SUBJECTS | MEANS (%) |
|---|---|
| 1 | 132.91 |
| 2 | 648.15 |
| 3 | −39.48 |
| TOTAL MEANS (%) | 247.19 |

It appears from the statistical analyses and graphs presented above that the placebo and homeopathic chlorella by themselves were not eliminating lead from the body.

The Homeopathic Chlorella+CGF+Cilantro compound was effective in different quantities and combinations. The most effective formulations were Group 6-Homeopathic chlorella (30%)+CGF (40%)+Cilantro (30%) Group at a quantity of 40 drops×3 times a day, which was eliminating 335.51% higher amounts of lead in the post-test urine after taking the treatment composition, compared to the baseline or pre-test urine.

Again, Group 6, the Homeopathic chlorella (30%)+CGF (40%)+Cilantro (30%) Group at a quantity of 60 drops×3 times a day was eliminating 466.47% higher amounts of lead in the post-test urine after taking the treatment composition, compared to the baseline or pre-test urine. So, the higher quantity of 60 drops×3 times a day was eliminating more lead in the urine than the 40 drops×3 times a day.

The highest amount of lead eliminated (142.16) in the faeces was for Group 4—Homeopathic chlorella (50%)+CGF (20%)+Cilantro (30%) at a quantity of 50 drops×3 times a day. It appears that lead prefers the urinary routes of elimination compared to the biliary routes via the faeces, as there is much more eliminated through the urine than the faeces.

### Arsenic in Urine

### Placebo or Control Group

Reviewing the changes in arsenic in urine pre- and post testing using the Placebo or Control Group whom were NOT taking any active substances apart from mineral water with a few drops of cognac added to mask the flavour. Ideally, there should have been no difference in the percentage of metals present in the urine pre and post testing, but there are always individual biochemical differences, which give somewhat different results for each person.

The graph below shows the percentage increase or decrease of arsenic in urine for the control group. There appears to be conformity with the exception of a couple of cases who had increased levels of arsenic in the post-test urine—at least one of these had continued working in the foundry for an extra day during the testing period so they were continually being exposed to arsenic from the work environment. These discrepancies add up to a mean increase of lead in urine in the post-test sample of 104.87% (see Case Summaries below), showing that the placebo was chelating arsenic from the body (but only in two of the 14 subjects).

FIG. 14 shows levels of Arsenic in Urine in the Control Group (60 drops×3 daily).

Case Summaries—the means represent the amount of metals increasing (+) or decreasing (−) in the post sample compared to the pre-sample.

### Placebo Group

60 drops×3 times a day

| SUBJECT | MEANS (%) |
|---|---|
| 1 | −7.14 |
| 2 | 263.16 |
| 3 | 87.50 |
| 4 | 13.33 |
| 5 | 37.50 |
| 6 | 6.25 |
| 7 | 33.33 |
| 8 | −55.56 |
| 9 | −21.43 |
| 10 | .00 |
| 11 | −28.57 |
| 12 | −6.67 |
| 13 | 1041.67 |
| TOTAL MEANS (%) | 104.87 |

### Homeopathic Protocol

The Homeopathic chlorella was tried by itself to see whether it was effective at detoxifying Arsenic in urine. Without the other synergistic ingredients, there was no increase in metals when comparing the pre and post-tests. Overall there was a −11.16% DECREASE in arsenic, indicating that the Homeopathic chlorella by itself was not chelating this metal. The quantities used were the maximal quantity of 60 drops×3 times a day and the trial was run over 24 hours.

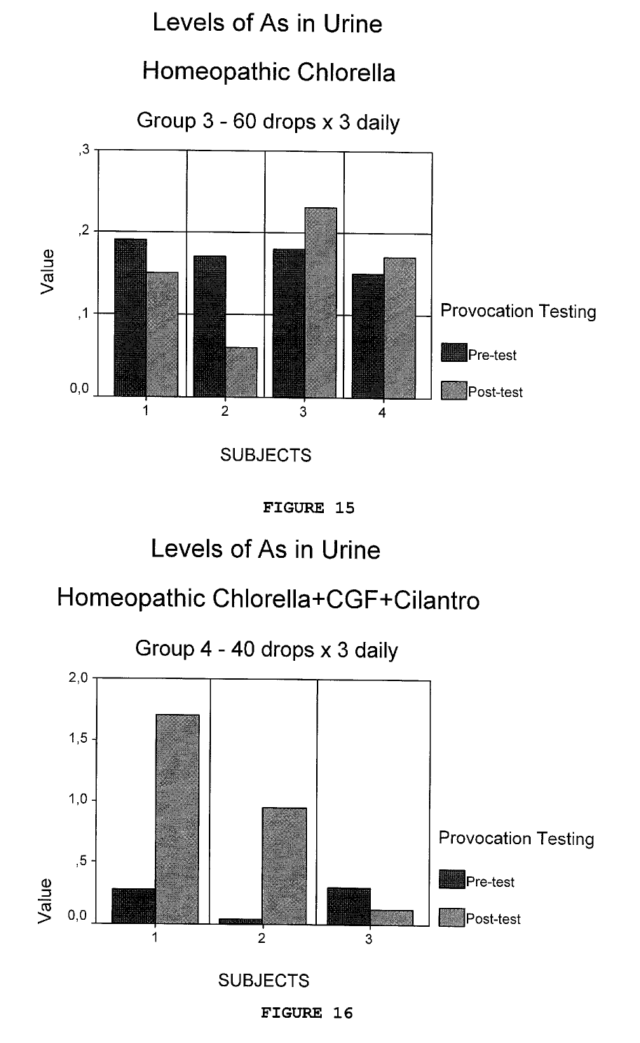

FIG. 15 shows levels of As in Urine in Group 3 (60 drops×3 daily) using Homeopathic chlorella.

Case Summaries—the means represent the amount of metals increasing (+) or decreasing (−) in the post sample compared to the pre-sample.

### Homeopathic Chlorella

60 drops×3 times a day

| SUBJECTS | MEANS (%) |
|---|---|
| 1 | −21.05 |
| 2 | −64.71 |
| 3 | 27.78 |
| 4 | 13.33 |
| MEAN TOTALS (%) | −11.16 |

### Effective Procedure Protocols

### Homeopathic Chlorella+CGF+Cilantro

The procedure protocol that worked most effectively for eliminating ARSENIC in URINE, based on this data, is the Homeopathic Chlorella+CGF+Cilantro. There were various quantities that were effective and we will examine these below. The graph below (Group 4) shows the 907.38% mean increase using a quantities of 40 drops×3 times a day (see Case Summaries) in arsenic in the post-procedure urine samples (Group 4=Homeopathic chlorella (50%)+CGF (20%)+Cilantro (30%).

FIG. 16 shows levels of As in Urine in Group 4 (40 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (50%)+CGF (20%)+Cilantro (30%)
Group 4—Arsenic in Urine—40 drops×3 times a day

| SUBJECTS | MEANS (%) |
|---|---|
| 1 | −60.00 |
| 2 | 507.14 |
| 3 | 2275.00 |
| MEAN TOTALS (%) | 907.38 |

The graph below (Group 4) shows the 784.18% mean increase (see Case Summaries) in arsenic in the post-procedure urine samples using 50 drops×3 times a day (Group 4=Homeopathic chlorella (50%)+CGF (20%)+Cilantro (30%).

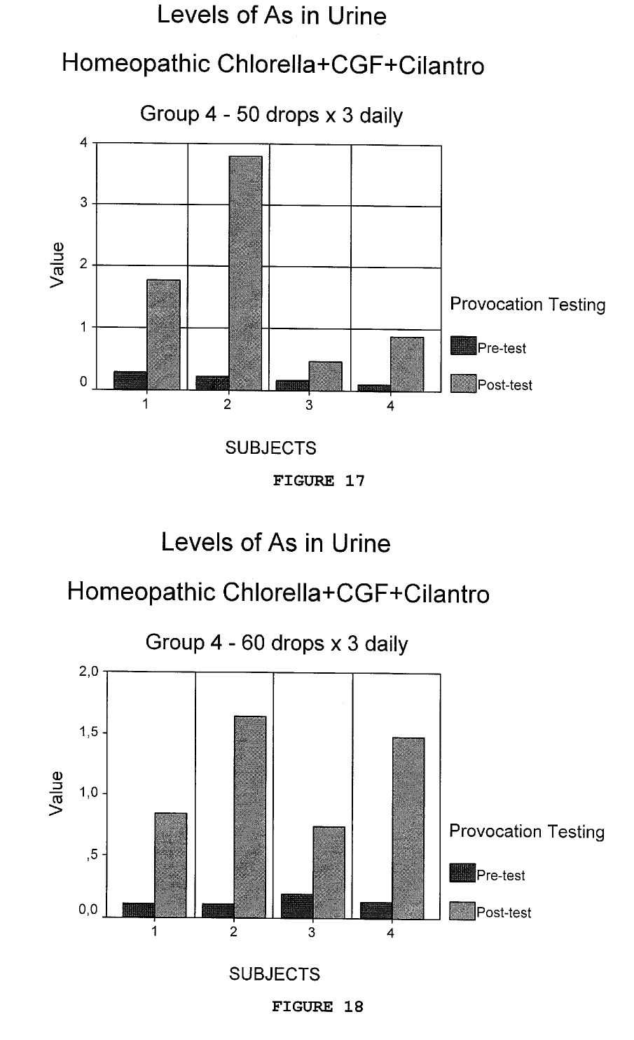

FIG. 17 shows levels in As in Urine in Group 4 (50 drops×3 daily) using Homeopathic chlorella, CGF and Cilantro.

Case Summaries—the means represent the amount of metals increasing (+) or decreasing (−) in the post sample compared to the pre-sample.

Group 4—Homeopathic chlorella (50%)+CGF (20%)+Cilantro (30%)
Arsenic in Urine—50 drops×3 times a day

| SUBJECTS | MEANS (%) |
|---|---|
| 1 | 535.71 |
| 2 | 780.00 |
| 3 | 1627.27 |
| 4 | 193.75 |
| MEAN TOTALS (%) | 784.18 |

The graph below (Group 4) shows the 841.10% mean increase (see Case Summaries) in arsenic in the post-procedure urine samples using 60 drops×3 times a day (Group 4=Homeopathic chlorella (50%)+CGF (20%)+Cilantro (30%).

FIG. 18 shows levels of As in Urine in Group 4 (60 drops×3 daily) using Homeopathic chlorella, CGF and Cilantro.

Case Summaries—the means represent the amount of metals increasing (+) or decreasing (−) in the post sample compared to the pre-sample.

Group 4—Homeopathic chlorella (50%)+CGF (20%)+Cilantro (30%)
Arsenic in Urine—60 drops×3 times a day

| SUBJECTS | MEANS (%) |
|---|---|
| 1 | 270.00 |
| 2 | 672.73 |
| 3 | 1390.91 |
| 4 | 1030.77 |
| MEAN TOTALS (%) | 841.10 |

The graph below (Group 5) shows the 776.33% mean increase (see Case Summaries) in arsenic in the post-procedure urine samples using 40 drops×3 times a day (Group 5=Homeopathic chlorella (40%)+CGF (30%)+Cilantro (30%).

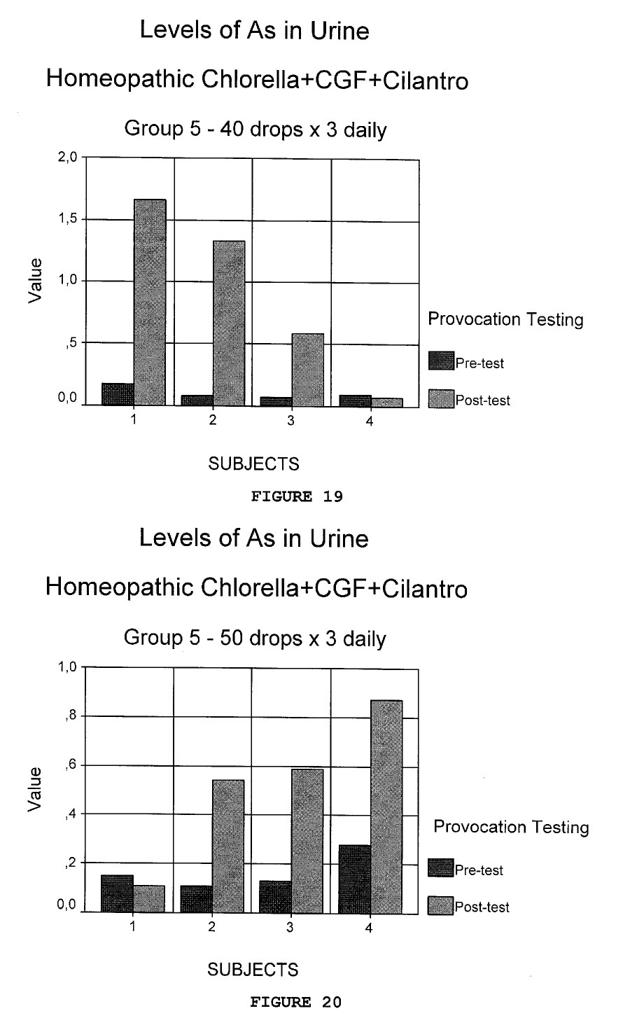

FIG. 19 shows levels of As in Urine in Group 5 (40 drops×3 daily) using Homeopathic chlorella, CGF and Cilantro.

Case Summaries—the means represent the amount of metals increasing (+) or decreasing (−) in the post sample compared to the pre-sample.

Group 5—Homeopathic chlorella (40%)+CGF (30%)+Cilantro (30%)
Arsenic in Urine—60 drops×3 times a day

| SUBJECTS | MEANS (%) |
|---|---|
| 1 | 822.22 |
| 2 | 1562.50 |
| 3 | 742.86 |
| 4 | −22.22 |
| MEAN TOTALS (%) | 776.33 |

The graph below (Group 5) shows the 232.20% mean increase (see Case Summaries) in arsenic in the post-procedure urine samples using 50 drops×3 times a day (Group 5=Homeopathic chlorella (40%)+CGF (30%)+Cilantro (30%).

FIG. 20 shows levels of As in Urine in Group 5 (50 drops×3 daily) using Homeopathic chlorella, CGF and Cilantro.

Case Summaries—the means represent the amount of metals increasing (+) or decreasing (−) in the post sample compared to the pre-sample.

Group 5—Homeopathic chlorella (40%)+CGF (30%)+Cilantro (30%)
Arsenic in Urine—50 drops×3 times a day

| SUBJECTS | MEANS (%) |
|---|---|
| 1 | −26.67 |
| 2 | 390.91 |
| 3 | 353.85 |
| 4 | 210.71 |
| MEAN TOTALS (%) | 232.20 |

The graph below (Group 5) shows the 1,440.39% mean increase (see Case Summaries) in arsenic in the post-procedure urine samples using 40 drops×3 times a day (Group 5=Homeopathic chlorella (40%)+CGF (30%)+Cilantro (30%)

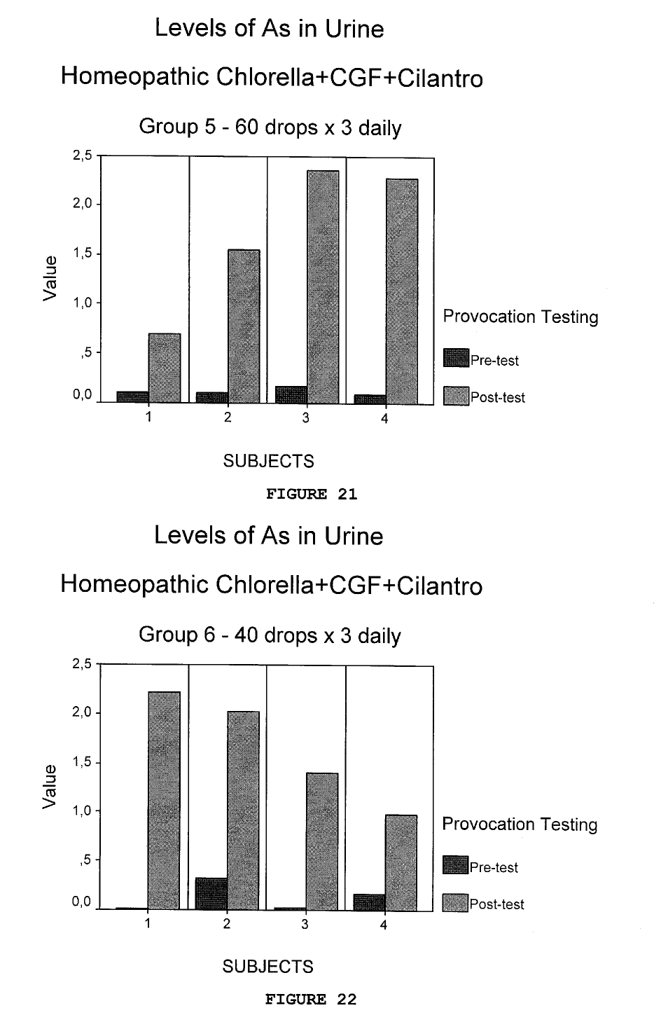

FIG. 21 shows levels of As in Urine in Group 5 (60 drops×3 daily) using Homeopathic chlorella, CGF and Cilantro.

Case Summaries—the means represent the amount of metals increasing (+) or decreasing (−) in the post sample compared to the pre-sample.

Group 5—Homeopathic chlorella (40%)+CGF (30%)+Cilantro (30%)
Arsenic in Urine—60 drops×3 times a day

| SUBJECTS (%) | MEANS (%) |
|---|---|
| 1 | 1450.00 |
| 2 | 2433.33 |
| 3 | 1288.24 |
| 4 | 590.00 |
| MEAN TOTALS (%) | 1,440.39 |

The graph below (Group 6) shows the 7,514.43% mean increase (see Case Summaries) in arsenic in the post-procedure urine samples using 40 drops×3 times a day (Group 6=Homeopathic chlorella (40%)+CGF (30%)+Cilantro (30%)

FIG. 22 shows levels of As in Urine in Group 6 (40 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%)
Arsenic in Urine—40 drops×3 times a day

| SUBJECTS | MEANS (%) |
|---|---|
| 1 | 476.47 |
| 2 | 22100.00 |
| 3 | 531.25 |
| 4 | 6950.00 |
| MEAN TOTALS (%) | 7,514.43 |

The graph below (Group 6) shows the 492.89% mean increase (see Case Summaries) in arsenic in the post-procedure urine samples using 50 drops×3 times a day (Group 6=Homeopathic chlorella (40%)+CGF (30%)+Cilantro (30%)

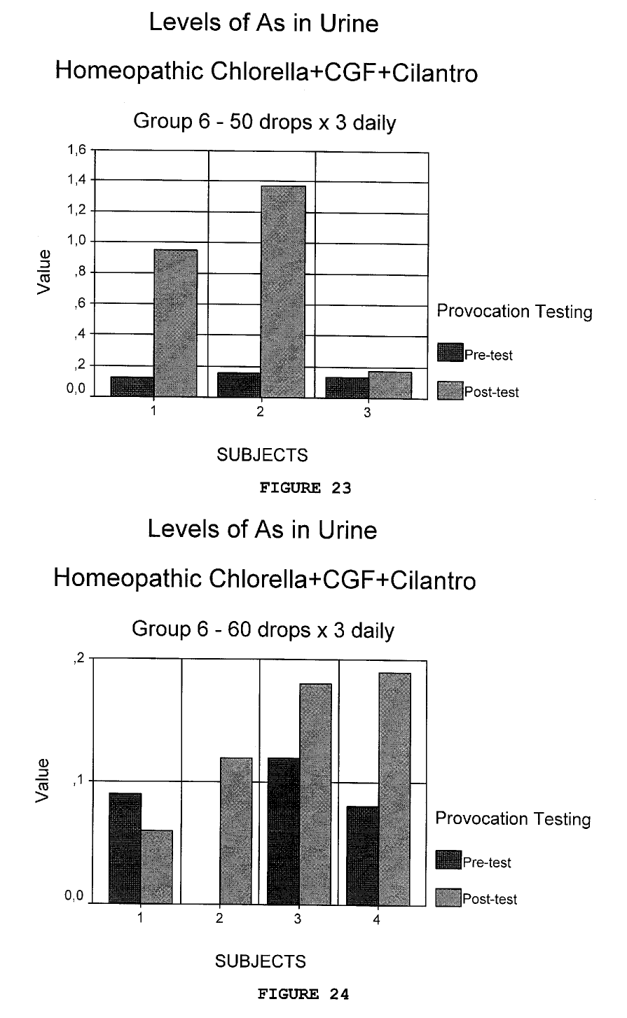

FIG. 23 shows levels of As in Urine in Group 6 (50 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%)
Arsenic in Urine—50 drops×3 times a day

| SUBJECTS | MEANS (%) |
|---|---|
| 1 | 691.67 |
| 2 | 756.25 |
| 3 | 30.77 |
| MEAN TOTALS (%) | 492.89 |

The graph below (Group 6) shows the 51.38% mean increase (see Case Summaries) in arsenic in the post-procedure urine samples using 60 drops×3 times a day (Group 6=Homeopathic chlorella (40%)+CGF (30%)+Cilantro (30%)

FIG. 24 shows levels of As in Urine in Group 6 (60 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%)
Arsenic in Urine—60 drops×3 times a day

| SUBJECTS | MEANS (%) |
|---|---|
| 1 | −33.33 |
| 2 | 50.00 |
| 3 | 137.50 |
| MEAN TOTALS (%) | 51.38 |

When Group 7 was tested using the Homeopathic chlorella (20%)+CGF (50%)+Cilantro (30%) there was not much elimination of arsenic in urine in the post-test compared to the pre-test.

### Arsenic in Faeces

### Placebo or Control Group

Let us begin by looking at the changes in arsenic in faeces pre- and post testing using the Placebo or Control Group whom were NOT taking any active substances apart from mineral water with a few drops of cognac added to mask the flavour. Ideally, there should have been no difference in the percentage of metals present in the faeces pre and post testing, but there are always individual biochemical differences, which give somewhat different results for each person.

The graph below shows the percentage increase or decrease of arsenic in faeces for the control group. There appear to be conformity with the exception of a couple of cases who had increased levels of arsenic in the post-test faeces—at least one of these had continued working in the foundry for an extra day during the testing period so they were continually being exposed to arsenic from the work environment. These discrepancies add tip to a mean increase of arsenic in faeces in the post-test sample of 61.13% (see Case Summaries below), showing that the placebo was chelating some arsenic in the faeces.

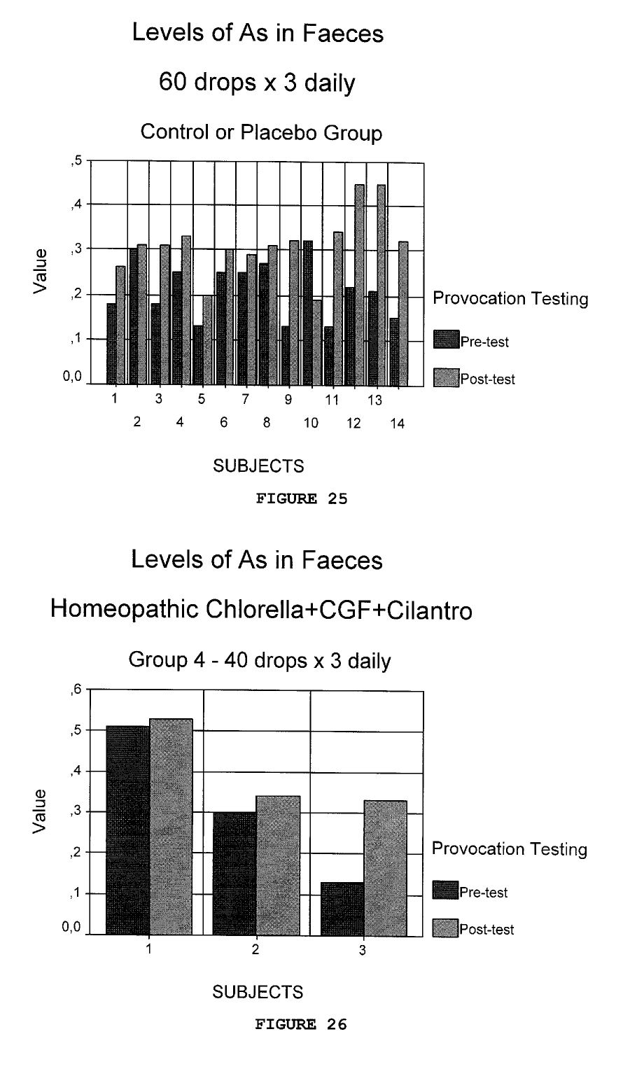

FIG. 25 shows levels of As in Faeces in the Control Group (60 drops×3 daily).

Case Summaries—the means represent the amount of metals increasing (+) or decreasing (−) in the post sample compared to the pre-sample.

### Placebo Group

60 drops×3 times a day

| SUBJECTS | MEANS (%) |
|---|---|
| 1 | 32.00 |
| 2 | 44.44 |
| 3 | 3.33 |
| 4 | 20.00 |
| 5 | 114.29 |
| 6 | 104.55 |
| 7 | 146.15 |
| 8 | 16.00 |
| 9 | 113.33 |
| 10 | 161.54 |
| 11 | 14.81 |
| 12 | −40.63 |
| 13 | 53.85 |
| 14 | 72.22 |
| MEAN TOTALS (%) | 61.13 |

### Homeopathic Protocol

The Homeopathic chlorella was tried by itself to see whether it was effective at detoxifying Arsenic in faeces. Without the other synergistic ingredients, there was no increase in metals when comparing the pre and post-tests. Overall there was a −18.51% DECREASE in arsenic, indicating that the Homeopathic chlorella by itself was not chelating this metal in faeces. The quantities used were the maximal quantity of 60 drops×3 times a day and the trial was run over 24 hours.

Case Summaries—the means represent the amount of metals increasing (+) or decreasing (−) in the post sample compared to the pre-sample.

### Homeopathic Chlorella Group

60 drops×3 times a day

| SUBJECTS | MEANS (%) |
|---|---|
| 1 | −36.11 |
| 2 | −27.78 |
| 3 | 8.33 |
| MEANS TOTALS (%) | −18.51 |

### Effective Procedure Protocols

### Homeopathic Chlorella+CGF+Cilantro

The procedure protocol that worked most effectively for eliminating ARSENIC in FAECES, based on this data, is the Homeopathic Chlorella+CGF+Cilantro. There were various quantities that were effective and we will examine these below. The graph below (Group 4) shows the 57.03% mean increase (see Case Summaries) in arsenic in the post-procedure faeces samples (Group 4=Homeopathic chlorella (50%)+CGF (20%)+Cilantro (30%).

FIG. 26 shows levels of As in Faeces in Group 4 (40 drops×3 daily) using Homeopathic chlorella, CGF and Cilantro.

Case Summaries—the means represent the amount of metals increasing (+) or decreasing (−) in the post sample compared to the pre-sample. _os Group 4—Homeopathic chlorella (50%)+CGF (20%)+Cilantro (30%).

40 drops×3 times a day

| SUBJECTS | MEANS (%) |
|---|---|
| 1 | 153.85 |
| 2 | 3.92 |
| 3 | 13.33 |
| MEAN TOTALS (%) | 57.03 |

The graph below (Group 4) shows the 163.76% mean increase (see Case Summaries) in arsenic in the post-procedure faeces samples (Group 4=Homeopathic chlorella (50%)+CGF (20%)+Cilantro (30%).

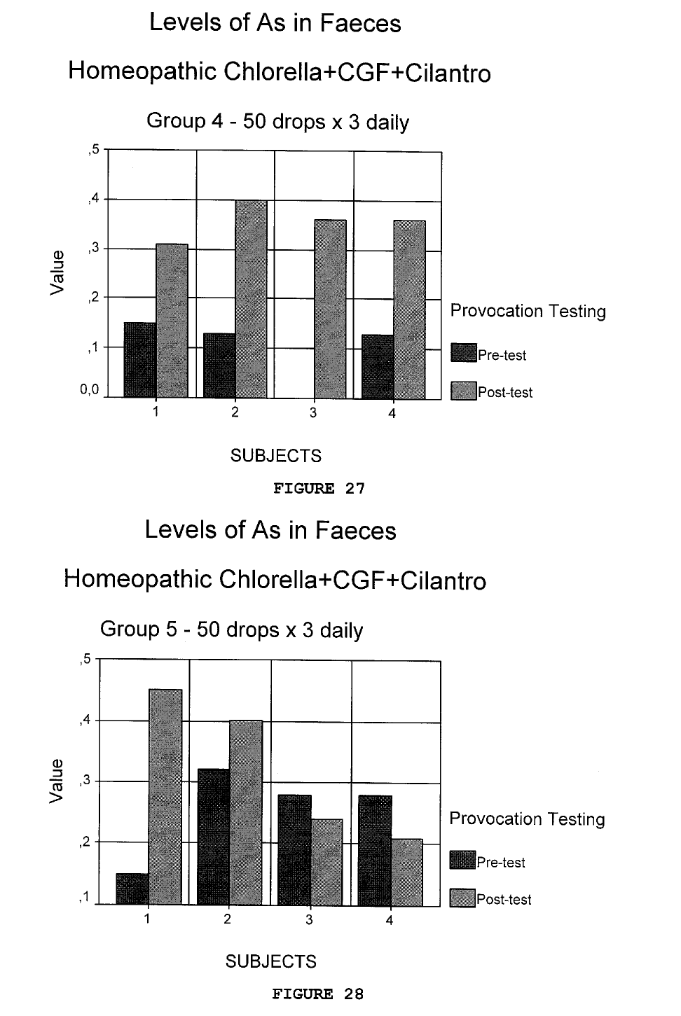

FIG. 27 shows levels of As in Faeces in Group 4 (50 drops×3 daily) using Homeopathic chlorella, CGF and Cilantro.

Case Summaries—the means represent the amount of metals increasing (+) or decreasing (−) in the post sample compared to the pre-sample.

Group 4—Homeopathic chlorella (50%)+CGF (20%)+Cilantro (30%).
50 drops×3 times a day

| SUBJECTS | MEANS (%) |
|---|---|
| 1 | 106.67 |
| 2 | 176.92 |
| 3 | 207.69 |
| MEAN TOTALS (%) | 163.76 |

The graph below (Group 5) shows the 46.42% mean increase (see Case Summaries) in arsenic in the post-procedure faeces samples (Group 5=Homeopathic chlorella (40%)+CGF (30%)+Cilantro (30%)

FIG. 28 shows levels of As in Faeces in Group 5 (50 drops×3 daily) using Homeopathic chlorella, CGF and Cilantro.

Case Summaries—the means represent the amount of metals increasing (+) or decreasing (−) in the post sample compared to the pre-sample.

Group 5—Homeopathic chlorella (40%)+CGF (30%)+Cilantro (30%)
50 drops×3 times a day

| SUBJECTS | MEANS (%) |
|---|---|
| 1 | 200.00 |
| 2 | 25.00 |
| 3 | −14.29 |
| 4 | −25.00 |
| MEAN TOTALS (%) | 46.42 |

The graph below (Group 6) shows the 52.32% mean increase (see Case Summaries) in arsenic in the post-procedure faeces samples (Group 6=Homeopathic chlorella (30%)+CGF (40%)+Cilantro (30%).

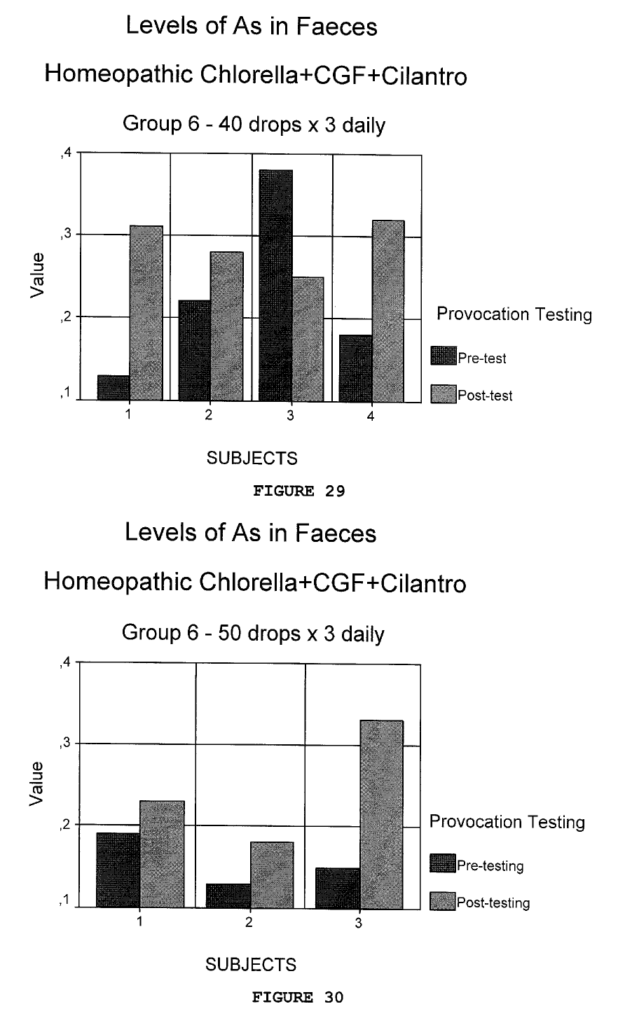

FIG. 29 shows levels of As in Faeces in Group 6 (40 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%)
40 drops×3 times a day

| SUBJECTS | MEANS (%) |
|---|---|
| 1 | 77.78 |
| 2 | 138.46 |
| 3 | 27.27 |
| 4 | −34.21 |
| MEAN TOTALS (%) | 52.32 |

The graph below (Group 6) shows the 59.83% mean increase (see Case Summaries) in arsenic in the post-procedure faeces samples (Group 6=Homeopathic chlorella (30%)+CGF (40%)+Cilantro (30%)

FIG. 30 shows levels of As in Faeces in Group 6 (50 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%)
50 drops×3 times a day

| SUBJECTS | MEANS (%) |
|---|---|
| 1 | 21.05 |
| 2 | 38.46 |
| 3 | 120.00 |
| MEAN TOTALS (%) | 59.83 |

The graph below (Group 7) shows the 69.43% mean increase (see Case Summaries) in arsenic in the post-procedure faeces samples (Group 7=Homeopathic chlorella (20%)+CGF (50%)+Cilantro (30%)

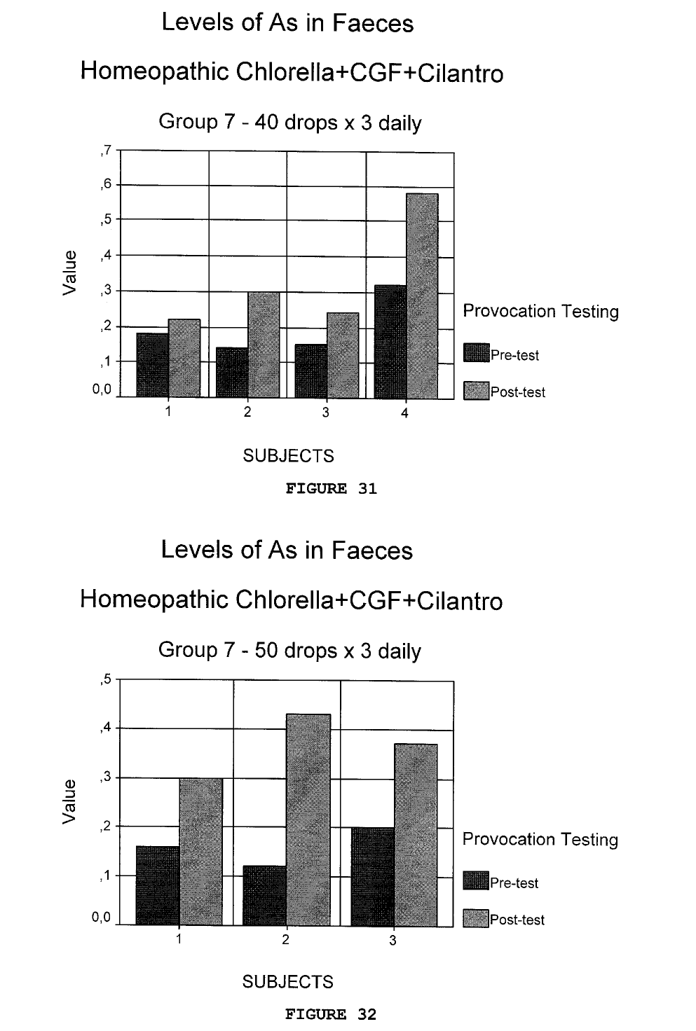

FIG. 31 shows levels of As in Faeces in Group 7 (40 drops×3 daily) using Homeopathic chlorella, CGF and Cilantro.

Case Summaries—the means represent the amount of metals increasing (+) or decreasing (−) in the post sample compared to the pre-sample.

Group 7—Homeopathic chlorella (20%)+CGF (50%)+Cilantro (30%)
40 drops×3 times a day

| SUBJECTS | MEANS (%) |
|---|---|
| 1 | 22.22 |
| 2 | 114.29 |
| 3 | 60.00 |
| 4 | 81.25 |
| MEAN TOTALS (%) | 69.43 |

The graph below (Group 7) shows the 143.61% mean increase (see Case Summaries) in arsenic in the post-procedure faeces samples (Group 7=Homeopathic chlorella (20%)+CGF (50%)+Cilantro (30%)

FIG. 32 shows levels of As in Faeces in Group 7 (50 drops×3 daily) using Homeopathic chlorella, CGF and Cilantro.

Case Summaries—the means represent the amount of metals increasing (+) or decreasing (−) in the post sample compared to the pre-sample.

Group 7—Homeopathic chlorella (20%)+CGF (50%)+Cilantro (30%)
50 drops×3 times a day

| SUBJECTS | MEANS (%) |
|---|---|
| 1 | 87.50 |
| 2 | 258.33 |
| 3 | 85.00 |
| MEAN TOTALS (%) | 143.61 |

The graph below (Group 7) shows the 298.10% mean increase (see Case Summaries) in arsenic in the post-procedure faeces samples (Group 7=Homeopathic chlorella (20%)+CGF (50%)+Cilantro (30%)

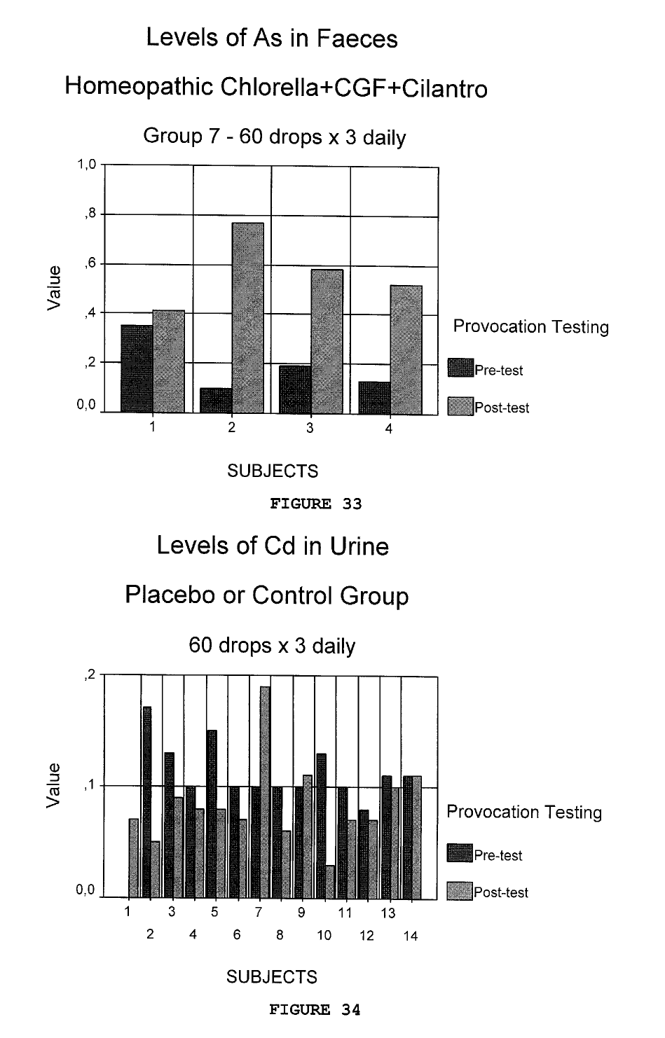

FIG. 33 shows levels in As in Faeces in Group 7 (60 drops×3 daily) using Homeopathic chlorella, CGF and Cilantro.

Case Summaries—the means represent the amount of metals increasing (+) or decreasing (−) in the post sample compared to the pre-sample.

Group 7—Homeopathic chlorella (20%)+CGF (50%)+Cilantro (30%)
60 drops×3 times a day

| SUBJECTS | MEANS (%) |
|---|---|
| 1 | 17.14 |
| 2 | 670.00 |
| 3 | 205.26 |
| 4 | 300.00 |
| MEAN TOTALS (%) | 298.10 |

### Summary & Conclusions

There were a number of effective protocols at different quantities and concentrations of the Homeopathic Chlorella+CGF+Cilantro as follows:

### Arsenic in Urine

1. Group 4—Homeopathic chlorella (50%)+CGF (20%)+Cilantro (30%)
40 drops×3 times a day—907.38%
2. Group 4—Homeopathic chlorella (50%)+CGF (20%)+Cilantro (30%)
50 drops×3 times a day—784.18%
3. Group 4—Homeopathic chlorella (50%)+CGF (20%)+Cilantro (30%)
60 drops×3 times a day—841.10%
4. Group 5—Homeopathic chlorella (40%)+CGF (30%)+Cilantro (30%)
40 drops×3 times a day—776.33%
5. Group 5—Homeopathic chlorella (40%)+CGF (30%)+Cilantro (30%)
50 drops×3 times a day—232.20%
6. Group 5—Homeopathic chlorella (40%)+CGF (30%)+Cilantro (30%)
60 drops×3 times a day—1,440.39%
7. Group 6—Homeopathic chlorella (30%)+CGF (40%)+Cilantro (30%)
40 drops×3 times a day—7,514.43%
8. Group 6—Homeopathic chlorella (30%)+CGF (40%)+Cilantro (30%)
50 drops×3 times a day—492.89%

### Arsenic in Faeces

1. Group 4—Homeopathic chlorella (50%)+CGF (20%)+Cilantro (30%).
50 drops×3 times a day—163.76%
2. Group 5—Homeopathic chlorella (40%)+CGF (30%)+Cilantro (30%)
50 drops×3 times a day—46.42%
3. Group 6—Homeopathic chlorella (30%)+CGF (40%)+Cilantro (30%)
40 drops×3 times a day—52.32%
4. Group 6—Homeopathic chlorella (30%)+CGF (40%)+Cilantro (30%)
50 drops×3 times a day—59.83%
5. Group 7—Homeopathic chlorella (20%)+CGF (50%)+Cilantro (30%)
40 drops×3 times a day—69.43%
6. Group 7—Homeopathic chlorella (20%)+CGF (50%)+Cilantro (30%)
50 drops×3 times a day—143.61%
7. Group 7—Homeopathic chlorella (20%)+CGF (50%)+Cilantro (30%)
60 drops×3 times a day—298.10%

It appears that arsenic prefers the urinary route of elimination, as opposed to the biliary route through the faeces.

Group 6—Homeopathic chlorella (30%)+CGF (40%)+Cilantro (30%) 40 drops×3 times a day—showed the highest elimination of lead in urine with a 7,514.43% increase in lead in the post-procedure group, when compared to the pre-procedure group.

It appears that the better route of elimination of arsenic is through the urinary route, which is again a big advantage in the sense that there is no re-absorption of heavy metals back into the body through the colon.

### Cadmium in Urine

### Placebo or Control Group

The changes of cadmium in urine pre- and post testing using the Placebo or Control Group who were NOT taking any active substances apart from mineral water with a few drops of cognac added to mask the flavour. Ideally, there should have been no difference in the percentage of metals present in the urine pre and post testing, but there are always individual biochemical differences, which give somewhat different results for each person.

The graph below shows the percentage increase or decrease of cadmium in urine for the control group. There appear to be conformity with the exception of one subject who had increased levels of cadmium in the post-test urine—this person had continued working in the foundry for an extra day during the testing period so they were continually being exposed to cadmium from the work environment. These discrepancies add up to a mean increase of cadmium in urine in the post-test sample of −20.50% (see Case Summaries below), showing that the placebo was not chelating cadmium from the body apart from the one exception.

FIG. 34 shows levels of Cd in Urine in the Control Group.

Case Summaries—the means represent the amount of metals increasing (+) or decreasing (−) in the post sample compared to the pre-sample.

### Placebo Group

60 drops×3 daily

| SUBJECTS | MEANS (%) |
|---|---|
| 1 | −20.00 |
| 2 | −70.59 |
| 3 | −30.00 |
| 4 | −9.09 |
| 5 | −12.50 |
| 6 | 10.00 |
| 7 | 90.00 |
| 8 | .00 |
| 9 | −30.00 |
| 10 | −40.00 |
| 11 | −76.92 |
| 12 | −46.67 |
| 13 | −30.77 |
| MEAN TOTALS (%) | −20.5029 |

### Homeopathic Protocol

The Homeopathic chlorella was tried by itself to see whether it was effective at detoxifying cadmium in urine. Without the other synergistic ingredients, there was no increase in metals when comparing the pre and post-tests. Overall there was a −27.91% DECREASE in cadmium, indicating that the Homeopathic chlorella by itself was not chelating this metal. There were varying dosages used from 40 drops×3 daily (−24.44%), the 50 drops×3 daily (see Case Summaries below) and the 60 drops×3 daily (−32.68%). None of these dosages were eliminating cadmium in urine.

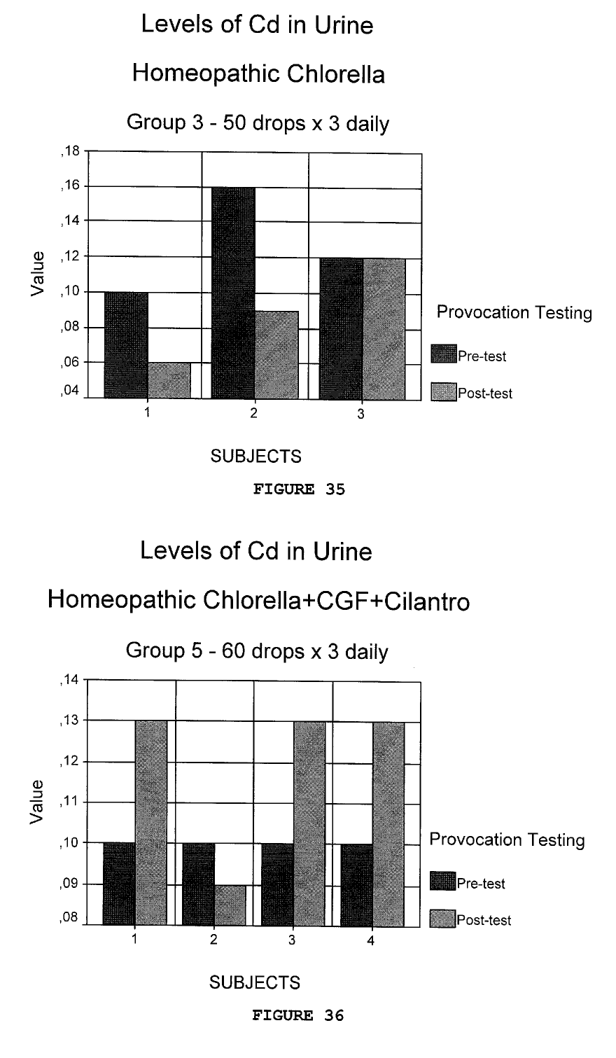

FIG. 35 shows levels of Cd in Urine in Group 3 (50 drops×3 daily) using Homeopathic chlorella.

Case Summaries—the means represent the amount of metals increasing (+) or decreasing (−) in the post sample compared to the pre-sample.

### Homeopathic Chlorella Group

50 drops×3 daily

| SUBJECTS | MEANS (%) |
|---|---|
| 1 | .00 |
| 2 | −40.00 |
| 3 | −43.75 |
| MEAN TOTALS (%) | −27.91 |

### Effective Treatment Protocols

### Homeopathic Chlorella+CGF+Cilantro

The treatment protocol that worked most effectively for eliminating CADMIUM in URINE, based on this data, is the Homeopathic Chlorella+CGF+Cilantro. The only dosage that was effective within this group was the 50 drops×3 daily—the other two dosages of 40 drops (−38.48) and the 60 drops×3 daily (−26.35) did not appear to be eliminating this metal in the urine. The graph below (Group 4) shows the 26.40% mean increase using a dosage of 50 drops×3 daily (see Case Summaries) in cadmium in the post-treatment urine samples (Group 4=Homeopathic chlorella (50%)+CGF (20%)+Cilantro (30%).

Case Summaries—the means represent the amount of metals increasing (+) or decreasing (−) in the post sample compared to the pre-sample.

GROUP 4—Homeopathic chlorella (50%)+CGF (20%)+Cilantro (30%)
50 drops×3 daily

| SUBJECTS | MEANS (%) |
|---|---|
| 1 | 27.27 |
| 2 | .00 |
| 3 | 20.00 |
| 4 | 58.33 |
| MEAN TOTALS (%) | 26.40 |

The graph below (Group 5) shows the 20.0% mean increase (see Case Summaries) in cadmium in the post-treatment urine samples using 60 drops×3 daily (Group 5=Homeopathic chlorella (40%)+CGF (30%)+Cilantro (30%). There was no elimination of this metal at the lower dosages of 40 and 50 drops×3 daily.

FIG. 36 shows levels of Cd in Urine in Group 5 (60 drops×3 daily) using Homeopathic chlorella, CGF and Cilantro.

Case Summaries—the means represent the amount of metals increasing (+) or decreasing (−) in the post sample compared to the pre-sample.

GROUP 5—Homeopathic chlorella (40%)+CGF (30%)+Cilantro (30%)
60 drops×3 daily

| SUBJECTS | MEANS (%) |
|---|---|
| 1 | −10.00 |
| 2 | 30.00 |
| 3 | 30.00 |
| 4 | 30.00 |
| MEAN TOTALS (%) | 20.00 |

The graph below (Group 6) shows the 22.50% mean increase (see Case Summaries) in cadmium in the post-treatment urine samples using 60 drops×3 daily (Group 6=Homeopathic chlorella (40%)+CGF (30%)+Cilantro (30%). There was no elimination of this metal at the lower dosages of 40 and 50 drops×3 daily.

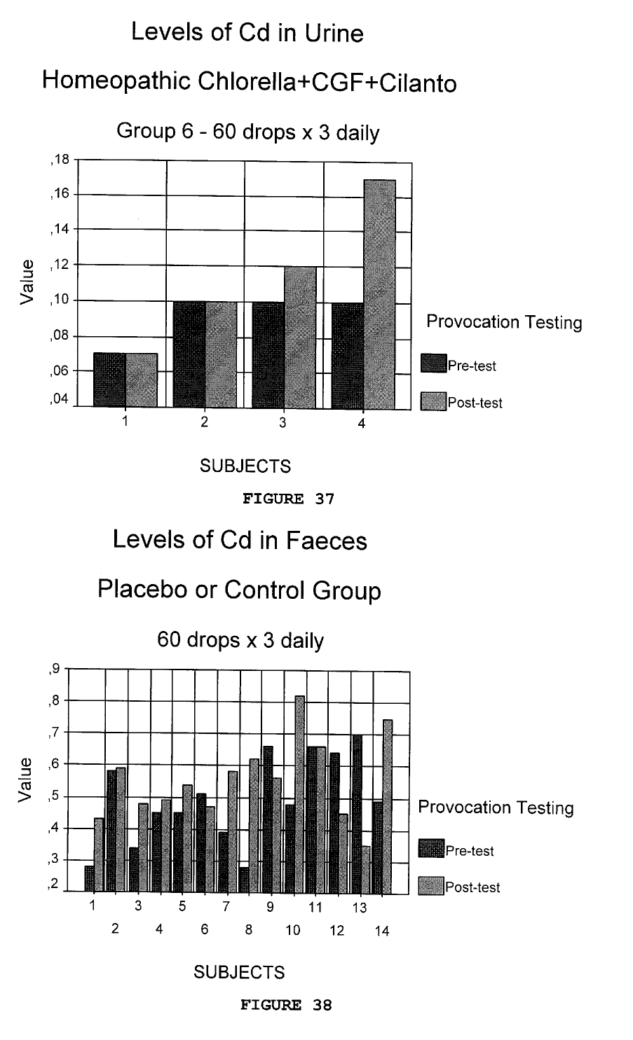

FIG. 37 shows levels of Cd in Urine in Group 6 (60 drops×3 daily) using Homeopathic chlorella, CGF and Cilantro.

Case Summaries—the means represent the amount of metals increasing (+) or decreasing (−) in the post sample compared to the pre-sample.

GROUP 6—Homeopathic chlorella (40%)+CGF (30%)+Cilantro (30%) 60 drops×3 daily

| SUBJECTS | MEANS (%) |
|---|---|
| 1 | .00 |
| 2 | .00 |
| 3 | 20.00 |
| 4 | 70.00 |
| MEAN TOTALS (%) | 22.50 |

### Cadmium in Faeces

### Placebo or Control Group

Let us begin by looking at the changes in cadmium in faeces pre- and post testing using the Placebo or Control Group whom were NOT taking any active substances apart from mineral water with a few drops of cognac added to mask the flavour. Ideally, there should have been no difference in the percentage of metals present in the faeces pre and post testing, but there are always individual biochemical differences, which give somewhat different results for each person.

The graph below shows the percentage increase or decrease of cadmium in faeces for the control group. There appear to be conformity with the exception of a couple of subjects who had increased levels of cadmium in the post-test faeces—at least one of these subjects had continued working in the foundry for an extra day during the testing period so they were continually being exposed to cadmium from the work environment. These discrepancies add up to a mean decrease of cadmium in faeces in the post-test sample of 22.62% (see Case Summaries below), showing that the placebo was chelating cadmium in a few subjects.

FIG. 38 shows levels of Cd in Faeces in the Control Group.

Case Summaries—the means represent the amount of metals increasing (+) or decreasing (−) in the post sample compared to the pre-sample.

### Placebo Group

60 drops×3 daily

| SUBJECTS | MEANS (%) |
|---|---|
| 1 | 8.89 |
| 2 | 53.57 |
| 3 | 1.72 |
| 4 | −7.84 |
| 5 | −50.00 |
| 6 | −29.69 |
| 7 | −15.15 |
| 8 | 48.72 |
| 9 | 53.06 |
| 10 | .00 |
| 11 | 121.43 |
| 12 | 70.83 |
| 13 | 20.00 |
| 14 | 41.18 |
| MEAN TOTALS (%) | 22.6228 |

### Homeopathic Protocol

The Homeopathic chlorella was tried by itself to see whether it was effective at detoxifying cadmium in faeces. Without the other synergistic ingredients, there was no increase in metals when comparing the pre and post-tests. Overall there was a 0.82% increase in cadmium, indicating that the Homeopathic chlorella by itself was not chelating this metal. The dosage used here was 60 drops×3 daily.

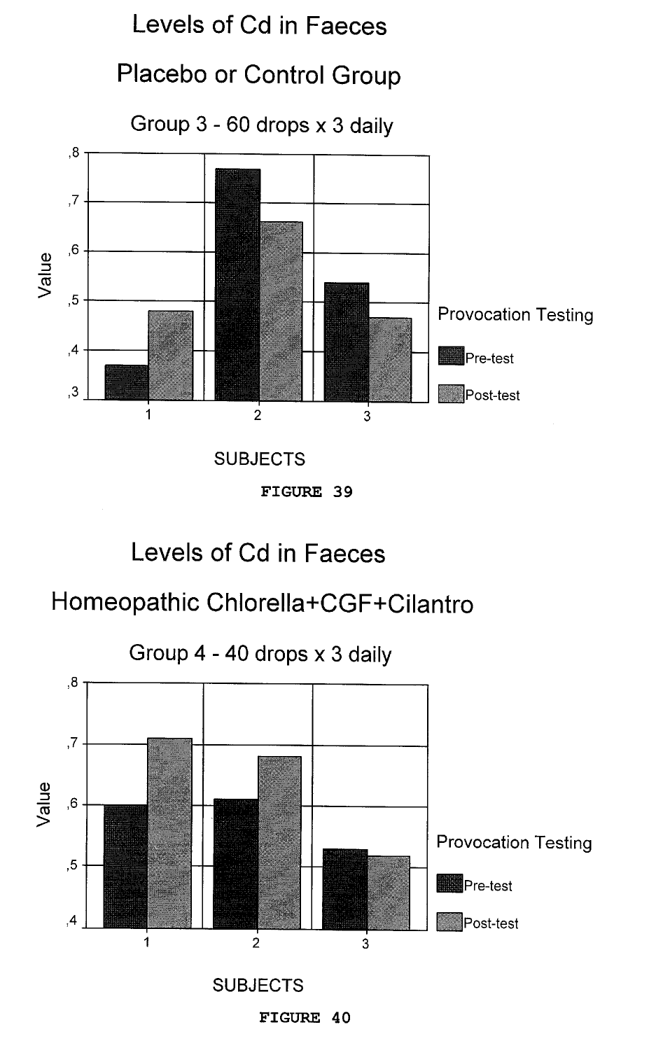

FIG. 39 shows levels of Cd in Faeces in the Control Group.

Case Summaries—the means represent the amount of metals increasing (+) or decreasing (−) in the post sample compared to the pre-sample.

### Homeopathic Chlorella

Group 3—60 drops×3 daily

| SUBJECTS | MEANS (%) |
|---|---|
| 1 | −12.96 |
| 2 | 29.73 |
| 3 | −14.29 |
| MEAN TOTALS (%) | 0.8270 |

### Effective Treatment Protocols

### Homeopathic Chlorella+CGF+Cilantro

The treatment protocol that worked most effectively for eliminating CADMIUM in FAECES, based on this data, is the Homeopathic Chlorella+CGF+Cilantro. The only dosage that was effective within this group was the 40 drops×3 daily—the other two dosages of 50 drops (1.39) and the 60 drops×3 daily (−10.32) did not appear to be eliminating this metal in the faeces. The graph below (Group 4) shows the 9.30% mean increase using a dosage of 40 drops×3 daily (see Case Summaries) in cadmium in the post-treatment faeces samples (Group 4=Homeopathic chlorella (50%)+CGF (20%)+Cilantro (30%).

FIG. 40 shows levels of Cd in Faeces in Group 4 (40 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (50%)+CGF (20%)+Cilantro (30%)
Group 4—40 drops×3 daily

| SUBJECTS | MEANS (%) |
|---|---|
| 1 | −1.89 |
| 2 | 18.33 |
| 3 | 11.48 |
| MEAN TOTALS (%) | 9.30 |

The graph below (Group 5) shows the 21.98% mean increase (see Case Summaries) in cadmium in the post-treatment faeces samples using 40 drops×3 daily (Group 5=Homeopathic chlorella (40%)+CGF (30%)+Cilantro (30%). There was no elimination of this metal at the dosages of 50 and 60 drops×3 daily.

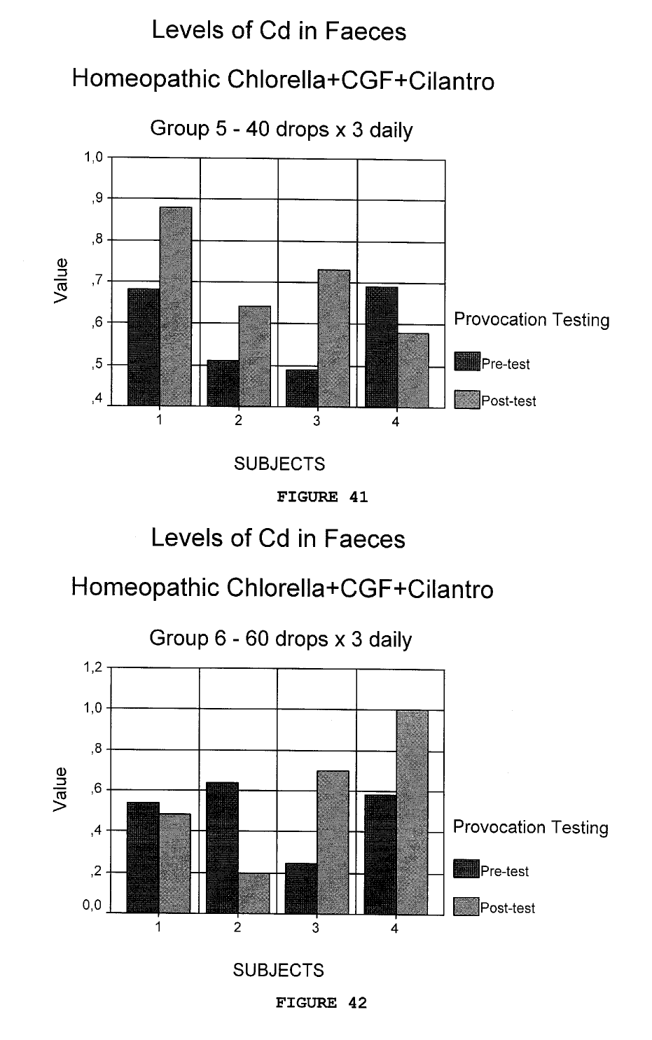

FIG. 41 shows levels of Cd in Faeces in Group 5 (40 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (50%)+CGF (20%)+Cilantro (30%)
Group 5—40 drops×3 daily

| SUBJECTS | MEANS (%) |
|---|---|
| 1 | 29.41 |
| 2 | 25.49 |
| 3 | 48.98 |
| 4 | −15.94 |
| MEAN TOTALS (%) | 21.98 |

The graph below (Group 6) shows the 43.13% mean increase (see Case Summaries) in cadmium in the post-treatment faeces samples using 60 drops×3 daily (Group 6=Homeopathic chlorella (30%)+CGF (40%)+Cilantro (30%).

There was no elimination of this metal at the dosages of 40 drops×3 daily, and only a slight elimination at 50 drops×3 daily (16.40).

FIG. 42 shows levels of Cd in Faeces in Group 6 (60 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (30%)+CGF (40%)+Cilantro (30%)
Group 6—60 drops×3 daily

| SUBJECTS | MEANS (%) |
|---|---|
| 1 | −11.11 |
| 2 | −68.75 |
| 3 | 180.00 |
| 4 | 72.41 |
| MEAN TOTALS (%) | 43.13 |

The graph below (Group 7) shows the 45.11% mean increase (see Case Summaries) in cadmium in the post-treatment faeces samples using 40 drops×3 daily (Group 7=Homeopathic chlorella (20%)+CGF (50%)+Cilantro (30%).

There was no elimination of this metal at the dosages of 50 drops and 60 drops×3 daily.

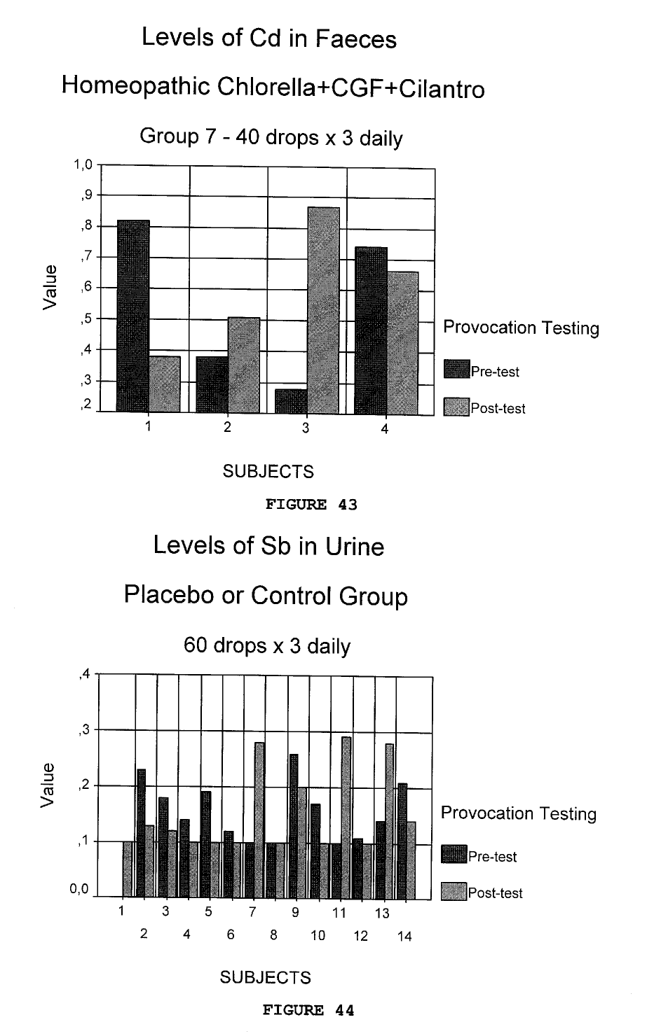

FIG. 43 shows levels of Cd in Faeces in Group 7 (40 drops×3 daily) using Homeopathic chlorella, CGF and Cilantro.

Case Summaries—the means represent the amount of metals increasing (+) or decreasing (−) in the post sample compared to the pre-sample.

### Homeopathic Chlorella (20%)+CGF (50%)+Cilantro (30%)

Group 7—40 drops×3 daily

| SUBJECTS | MEANS (%) |
|---|---|
| 1 | −53.66 |
| 2 | 34.21 |
| 3 | 210.71 |
| 4 | −10.81 |
| MEAN TOTALS (%) | 45.11 |

### Summary & Conclusions

The Placebo and the Homeopathic Chlorella did not appear to be eliminating cadmium in urine and faeces when used alone.

### Cadmium in Urine

The group that was eliminating the maximum amount of cadmium in urine was GROUP 4—Homeopathic chlorella (50%)+CGF (20%)+Cilantro (30%) at a dosage of 50 drops×3 daily—the total elimination was 26.4%.

### Cadmium in Faeces

The two groups that were most effective at eliminating cadmium in the faeces were:

1. Group 6—Homeopathic chlorella (30%)+CGF (40%)+Cilantro (30%) at a dosage of 60 drops×3 daily—43.13%.
2. Group 7—Homeopathic Chlorella (20%)+CGF (50%)+Cilantro (30%) 40 drops×3 daily—45.11%.

It appears that the better route of elimination of Cadmium is through the biliary route in the faeces.

### Antimony in Urine

### Placebo or Control Group

Let us examine the changes in antimony in urine pre- and post testing using the Placebo or Control Group who were NOT taking any active substances apart from mineral water with a few drops of cognac added to mask the flavour. Ideally, there should have been no difference in the percentage of metals present in the faeces pre and post testing, but there are always individual biochemical differences, which give somewhat different results for each person.

The graph below shows the percentage increase or decrease of antimony in urine for the control group. There appear to be conformity with the exception of a couple of subjects who had increased levels of antimony in the post-test urine—at least one of these subjects had continued working in the foundry for an extra day during the testing period so they were continually being exposed to antimony from the work environment. These discrepancies add up to a mean decrease of antimony in urine in the post-test sample of 14.91% (see Case Summaries below), showing that the placebo was chelating antimony in a few subjects.

FIG. 44 shows levels of Sb in Urine in the Control Group.

Case Summaries—the means represent the amount of metals increasing (+) or decreasing (−) in the post sample compared to the pre-sample.

### PLACEBO or CONTROL GROUP

60 drops×3 daily

| SUBJECTS | MEANS (%) |
|---|---|
| 1 | −28.57 |
| 2 | −43.48 |
| 3 | −16.67 |
| 4 | 100.00 |
| 5 | −9.09 |
| 6 | −23.08 |
| 7 | 180.00 |
| 8 | −33.33 |
| 9 | 190.00 |
| 10 | .00 |
| 11 | −41.18 |
| 12 | −47.37 |
| 13 | −33.33 |
| MEAN TOTALS (%) | 14.91 |

### Results of Heavy Metal Detoxification Protocols

### Homeopathic Protocol

The Homeopathic chlorella was tried by itself to see whether it was effective at detoxifying antimony in urine. Without the other synergistic ingredients, there was no increase in metals when comparing the pre and post-tests. Overall there was a −2.79% DECREASE in arsenic, indicating that the Homeopathic chlorella by itself was not chelating this metal in urine. The dosage used was the maximal dose of 60 drops×3 daily and the trial was run over 24 hours.

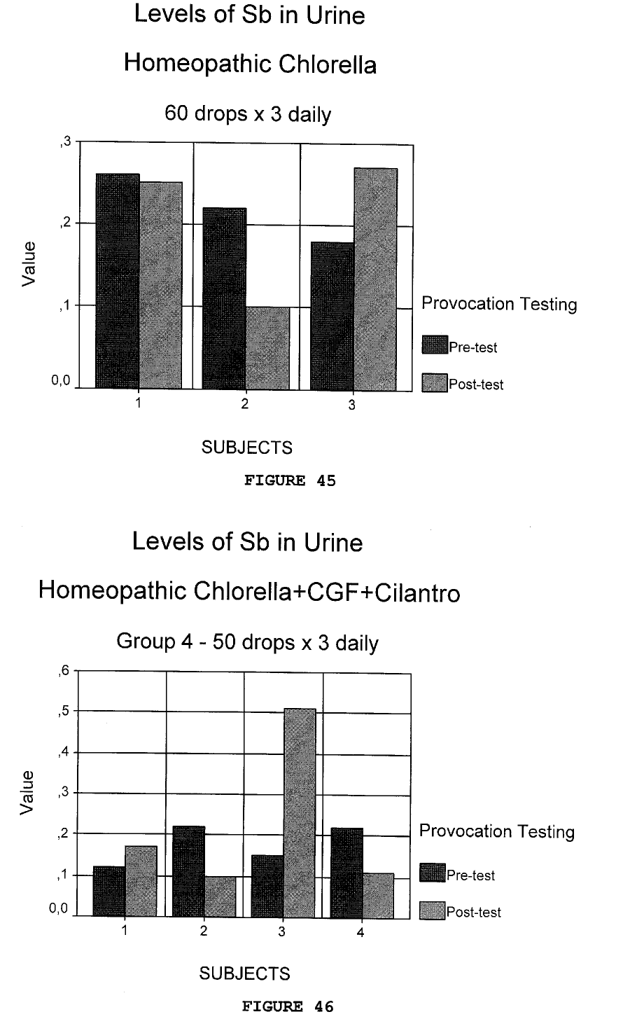

FIG. 45 shows levels of Sb in Urine (60 drops×3 daily) using Homeopathic chlorella.

Case Summaries—the means represent the amount of metals increasing (+) or decreasing (−) in the post sample compared to the pre-sample.

### Homeopathic Chlorella Group

60 drops×3 daily

| SUBJECTS | MEANS (%) |
|---|---|
| 1 | −54.55 |
| 2 | −54.5455 |
| 3 | −3.85 |
| 4 | −3.8462 |
| 5 | 50.00 |
| 6 | 50.0000 |
| MEAN TOTALS (%) | −2.79 |

### Effective Treatment Protocols

### Homeopathic Chlorella+CGF+Cilantro

The treatment protocol that worked most effectively for eliminating ANTIMONY in URINE, based on this data, is the Homeopathic Chlorella+CGF+Cilantro. There were various dosages that were effective and we will examine these below. The graph below (Group 4) shows the 44.28% mean increase (see Case Summaries) in antimony in the post-treatment urine samples (Group 4=Homeopathic chlorella (50%)+CGF (20%)+Cilantro (30%) at a dosage of 50 drops×3 daily.

FIG. 46 shows levels of Sb in Urine in Group 4 (50 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (50%)+CGF (20%)+Cilantro (30%)
Group 4—50 drops×3 daily

| SUBJECTS | MEANS (%) |
|---|---|
| 1 | 41.67 |
| 2 | −50.00 |
| 3 | −54.55 |
| 4 | 240.00 |
| MEAN TOTALS (%) | 44.28 |

The graph below (Group 5) shows the 22.87% mean increase (see Case Summaries) in antimony in the post-treatment urine samples (Group 5=Homeopathic chlorella (40%)+CGF (30%)+Cilantro (30%) at a dosage of 40 drops×3 daily.

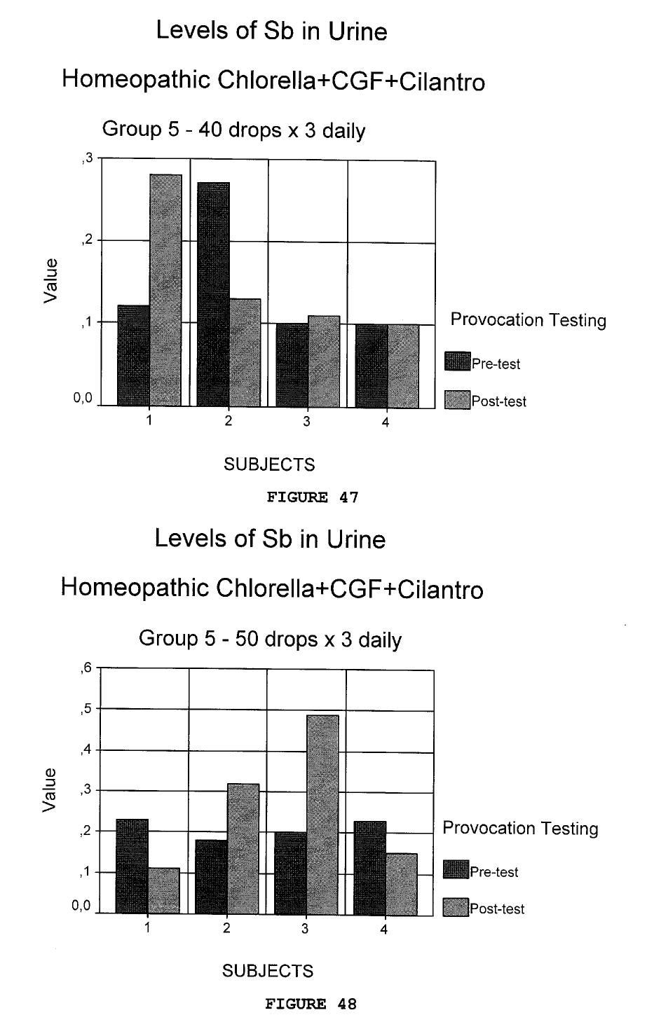

FIG. 47 shows levels of Sb in Urine in Group 5 (40 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (50%)+CGF (20%)+Cilantro (30%)
Group 5—40 drops×3 daily

| SUBJECTS | MEANS (%) |
|---|---|
| 1 | 133.33 |
| 2 | −51.85 |
| 3 | 10.00 |
| 4 | .00 |
| MEAN TOTALS (%) | 22.87 |

The graph below (Group 5) shows the 33.95% mean increase (see Case Summaries) in antimony in the post-treatment urine samples (Group 5=Homeopathic chlorella (40%)+CGF (30%)+Cilantro (30%) at a dosage of 50 drops×3 daily.

FIG. 48 shows levels of Sb in Urine in Group 5 (50 drops×3 daily) using Homeopathic chlorella, CGF and Cilantro.

Case Summaries—the means represent the amount of metals increasing (+) or decreasing (−) in the post sample compared to the pre-sample.

Homeopathic chlorella (50%)+CGF (20%)+Cilantro (30%)
Group 5—50 drops×3 daily

| SUBJECTS | MEANS (%) |
|---|---|
| 1 | −52.17 |
| 2 | 77.78 |
| 3 | 145.00 |
| 4 | −34.78 |
| MEAN TOTALS (%) | 33.95 |

The graph below (Group 6) shows the 48.83% mean increase (see Case Summaries) in antimony in the post-treatment urine samples (Group 6=Homeopathic chlorella (30%)+CGF (40%)+Cilantro (30%) Group at a dosage of 40 drops×3 daily.

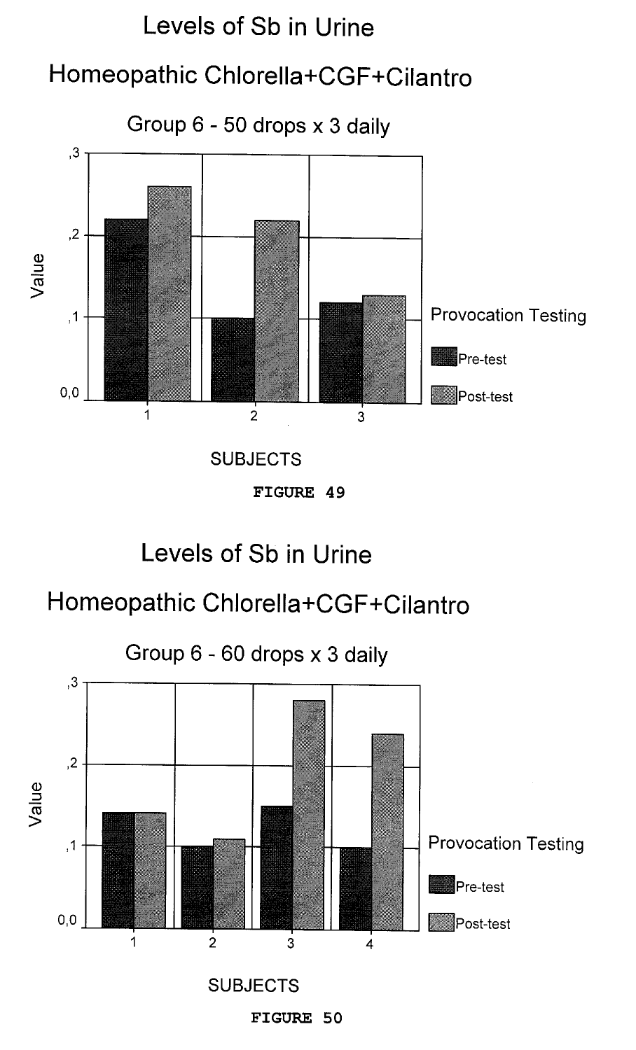

FIG. 49 shows levels of Sb in Urine in Group 6 (50 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%) Group 50 drops×3 daily

| SUBJECTS | MEANS (%) |
|---|---|
| 1 | 18.18 |
| 2 | 120.00 |
| 3 | 8.33 |
| MEAN TOTALS (%) | 48.83 |

The graph below (Group 6) shows the 59.15% mean increase (see Case Summaries) in antimony in the post-treatment urine samples (Group 6=Homeopathic chlorella (30%)+CGF (40%)+Cilantro (30%) Group at a dosage of 60 drops×3 daily.

FIG. 50 shows levels of Sb in Urine in Group 6 (60 drops×3 daily) using Homeopathic chlorella, CGF and Cilantro.

Case Summaries—the means represent the amount of metals increasing (+) or decreasing (−) in the post sample compared to the pre-sample.

Group 6—Homeopathic chlorella (30%)+CGF (40%)+Cilantro (30%) Group
60 drops×3 daily

| SUBJECTS | MEANS (%) |
|---|---|
| 1 | .00 |
| 2 | 10.00 |
| 3 | 86.67 |
| 4 | 140.00 |
| MEAN TOTALS (%) | 59.16 |

The graph below (Group 7) shows the 60.70% mean increase (see Case Summaries) in antimony in the post-treatment urine samples (Group 7=Homeopathic chlorella (20%)+CGF (50%)+Cilantro (30%) Group at a dosage of 40 drops×3 daily.

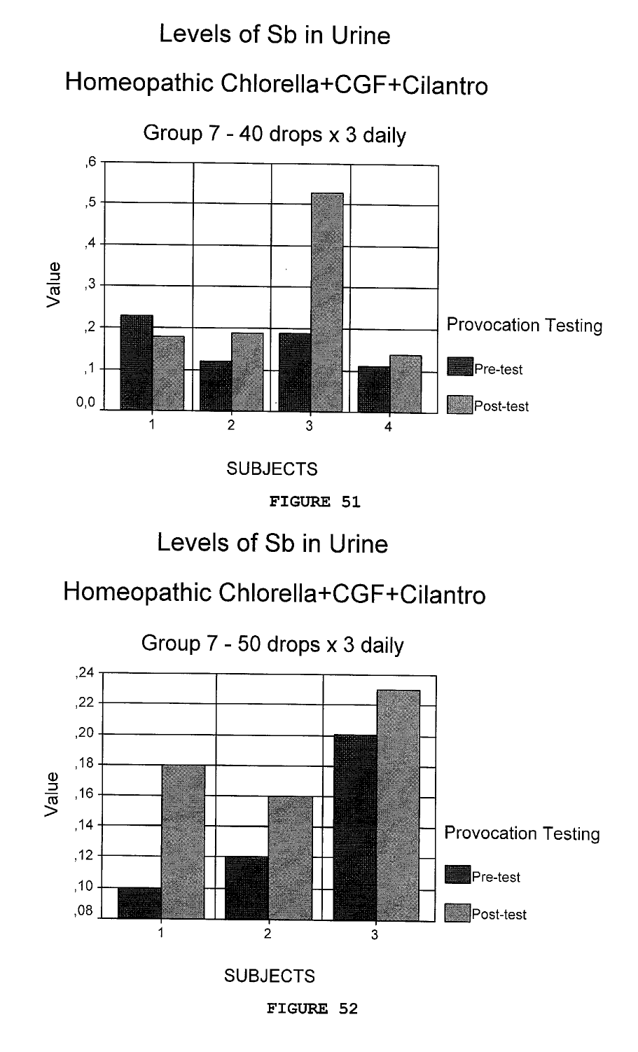

FIG. 51 shows levels of Sb in Urine in Group 7 (40 drops×3 daily) using Homeopathic chlorella, CGF and Cilantro.

Case Summaries—the means represent the amount of metals increasing (+) or decreasing (−) in the post sample compared to the pre-sample.

Group 7—Homeopathic chlorella (20%)+CGF (50%)+Cilantro (30%)
40 drops×3 daily

| SUBJECTS | MEANS (%) |
|---|---|
| 1 | −21.74 |
| 2 | 58.33 |
| 3 | 178.95 |
| 4 | 27.27 |
| MEAN TOTALS (%) | 60.70 |

The graph below (Group 7) shows the 42.77% mean increase (see Case Summaries) in antimony in the post-treatment urine samples (Group 7=Homeopathic chlorella (20%)+CGF (50%)+Cilantro (30%) Group at a dosage of 50 drops×3 daily.

FIG. 52 shows levels of Sb in Urine in Group 7 (50 drops×3 daily) using Homeopathic chlorella, CGF and Cilantro.

Case Summaries—the means represent the amount of metals increasing (+) or decreasing (−) in the post sample compared to the pre-sample.

Group 7—Homeopathic chlorella (20%)+CGF (50%)+Cilantro (30%)
50 drops×3 daily

| SUBJECTS | MEANS (%) |
|---|---|
| 1 | 80.00 |
| 2 | 33.33 |
| 3 | 15.00 |
| MEAN TOTALS (%) | 42.77 |

### Antimony in Faeces

### Placebo or Control Group

Let us begin by looking at the changes in antimony in faeces pre- and post testing using the Placebo or Control Group whom were NOT taking any active substances apart from mineral water with a few drops of cognac added to mask the flavour. Ideally, there should have been no difference in the percentage of metals present in the faeces pre and post testing, but there are always individual biochemical differences, which give somewhat different results for each person.

The graph below shows the percentage increase or decrease of antimony in faeces for the control group. There appear to be conformity with the exception of a couple of subjects who had increased levels of antimony in the post-test faeces—at least one of these subjects had continued working in the foundry for an extra day during the testing period so they were continually being exposed to antimony from the work environment. These discrepancies add up to a mean decrease of antimony in faeces in the post-test sample of 6.61% (see Case Summaries below), showing that the placebo was chelating antimony in two subjects.

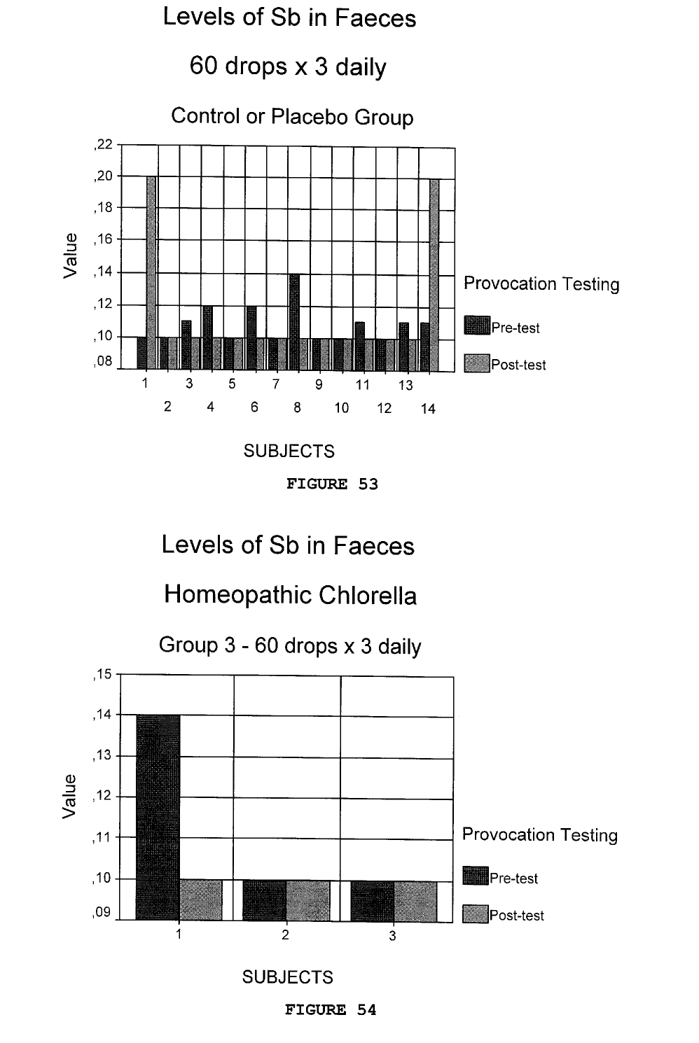

FIG. 53 shows levels of Sb in Faeces in the Control Group (60 drops×3 daily).

Case Summaries—the means represent the amount of metals increasing (+) or decreasing (−) in the post sample compared to the pre-sample.

### Placebo or Control Group

60 drops×3 daily

| SUBJECTS | MEANS (%) |
|---|---|
| 1 | −16.67 |
| 2 | 100.00 |
| 3 | .00 |
| 4 | −16.67 |
| 5 | −9.09 |
| 6 | .00 |
| 7 | .00 |
| 8 | .00 |
| 9 | 81.82 |
| 10 | −9.09 |
| 11 | −28.57 |
| 12 | .00 |
| 13 | .00 |
| 14 | −9.09 |
| MEAN TOTALS (%) | 6.61 |

### Homeopathic Protocol

The Homeopathic chlorella was tried by itself to see whether it was effective at detoxifying antimony in faeces. Without the other synergistic ingredients, there was no increase in metals when comparing the pre and post-tests. Overall there was a −9.52% DECREASE in arsenic, indicating that the Homeopathic chlorella by itself was not chelating this metal in faeces. The dosage used was the maximal dose of 60 drops×3 daily and the trial was run over 24 hours.

FIG. 54 shows levels of Sb in Faeces in Group 3 (60 drops×3 daily) using Homeopathic chlorella.

Case Summaries—the means represent the amount of metals increasing (+) or decreasing (−) in the post sample compared to the pre-sample.

### Homeopathic Chlorella Group

Group 3—60 drops×3 daily

| SUBJECTS | MEANS (%) |
|---|---|
| 1 | .00 |
| 2 | −28.57 |
| 3 | .00 |
| MEAN TOTALS (%) | −9.52 |

### Effective Treatment Protocols

### Homeopathic Chlorella+CGF+Cilantro

The treatment protocol that worked most effectively for eliminating ANTIMONY in FAECES, based on this data, is the Homeopathic Chlorella+CGF+Cilantro. There were various dosages that were effective and we will examine these below. The graph below (Group 5) shows the 50.00% mean increase (see Case Summaries) in antimony in the post-treatment faeces samples (Group 5=Homeopathic chlorella (40%)+CGF (30%)+Cilantro (30%)

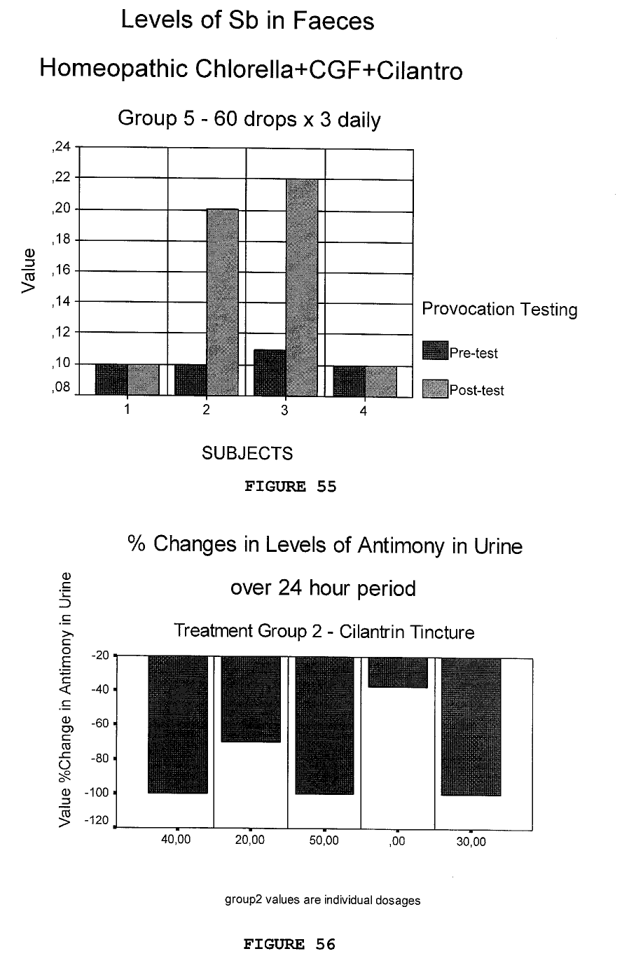

FIG. 55 shows levels of Sb in Faeces in Group 5 (60 drops×3 daily) using Homeopathic chlorella, CGF and Cilantro.

Case Summaries—the means represent the amount of metals increasing (+) or decreasing (−) in the post sample compared to the pre-sample.

### Homeopathic Chlorella+CGF+Cilantro Group

Group 5—60 drops×3 daily

| SUBJECTS | MEANS (%) |
|---|---|
| 1 | 100.00 |
| 2 | .00 |
| 3 | 100.00 |
| 4 | .00 |
| MEAN TOTALS (%) | 50.00 |

### Summary & Conclusions

The Placebo and Homeopathic Chlorella Groups did not demonstrate any elimination of antimony in either urine or faeces. By themselves, the placebo and homeopathic chlorella cannot eliminate antimony in the body.

If we examine a summary of the various compounds that were successful at eliminating antimony in urine we find quite a number of different dosages and strengths that were successful:

Group 4—50×3 drops—44.28%
Group 5—40×3 drops—22.87%
Group 5—50×3 drops—33.95%
Group 6—50×3 drops—48.83%
Group 6—60 drops×3—59.15%
Group 7—40×3 drops—60.70%
Group 7—50×3 daily—42.77%

It appears that the compound that eliminates the most antimony in urine is Group 6 at 60 drops×3 daily (59.15%) and Group 7 at 40 drops×3 daily (60.70%). The preferred elimination route is the urinary route through the urine as opposed to the faeces.

The compound that eliminated antimony in the faeces was Group 5 at 60 drops×3 daily (50%).

### Cilantro Tincture Alone

Cilantro or Cilantrin as it is sometime referred to is a tincture made from the Corriander sativum otherwise known as Chinese Parsley.

The Cilantro group consisted of 12 people in total that took 60 drops×3 daily.

This is an interesting group that showed the highest percentage DECREASE in metals—basically the percentage of metals had decreased between the pre-test and the post-test in both urine and faeces. The consistency of the results is indicative that the body is holding-on to the metals and probably storing in different cells and tissues, which is not the desired effect. Interestingly, when Cilantrin is combined with other detoxification substances such as Chlorella Growth Factor and Homeopathic Chlorella the result is completely the opposite, that is there is a percentage INCREASE of metals in both urine and faeces. The results are shown below:

Antimony in Urine—it is clear from the graph below that for all dosages there is a % DECREASE between the pre- and post-tests—this is indicative that the body is retaining the metals and not allowing them to be released.

FIG. 56 shows the percentage changes in levels of Antimony in Urine over a 24 hour period in Treatment Group 2 using Cilantrin Tincture.

Antimony in Faeces—the levels of antimony again show a percentage DECREASE for all dosages.

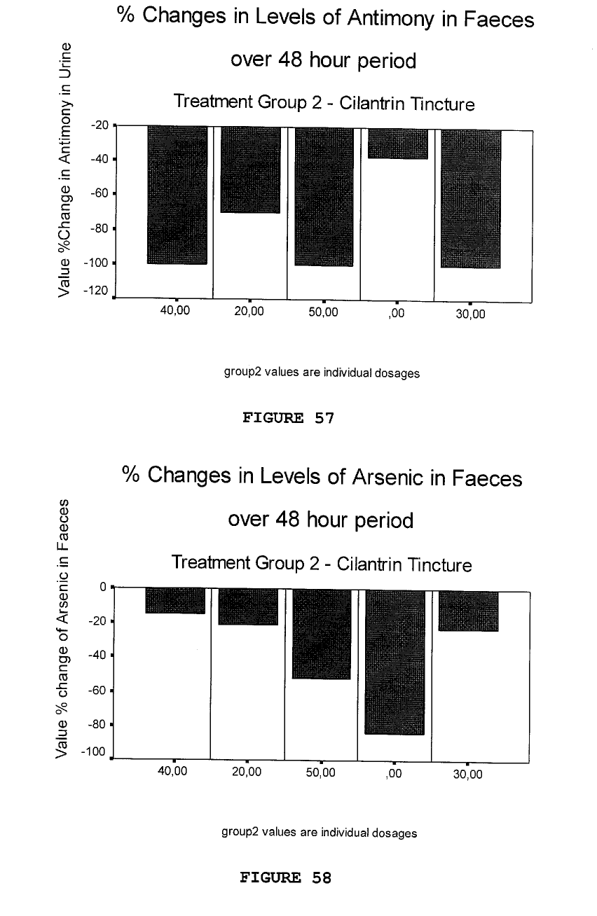

FIG. 57 shows the percentage changes in levels of Antimony in Faeces over a 48 hour period in Treatment Group 2 using Cilantrin Tincture.

Arsenic in Faeces—levels of arsenic show a percentage DECREASE for all dosages.

FIG. 58 shows the percentage changes in levels in Arsenic in Faeces over a 48 hour period in Treatment Group 2 using Cilantrin Tincture.

Arsenic in Urine—level of arsenic show % DECREASE for all dosages.

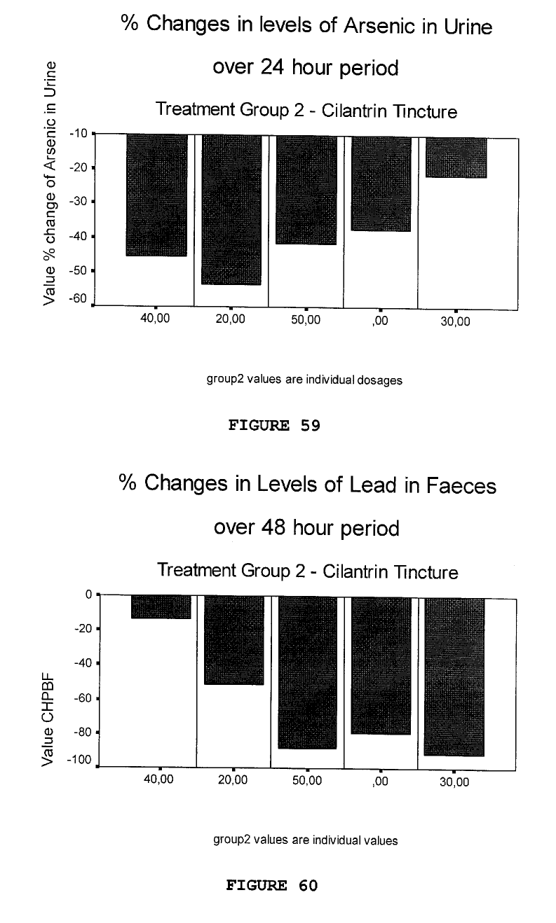

FIG. 59 shows percentage changes in levels of Lead in Faeces over a 24 hour period in Treatment Group 2 using Cilantrin Tincture.

Lead in Faeces—the same pattern emerges—a % DECREASE for all dosages.

FIG. 60 shows percentage changes in levels of Lead in Faeces over a 48 hour period in Treatment Group 2 using Cilantrin Tincture.

Lead in Urine—shows clearly a % DECREASE for all dosages.

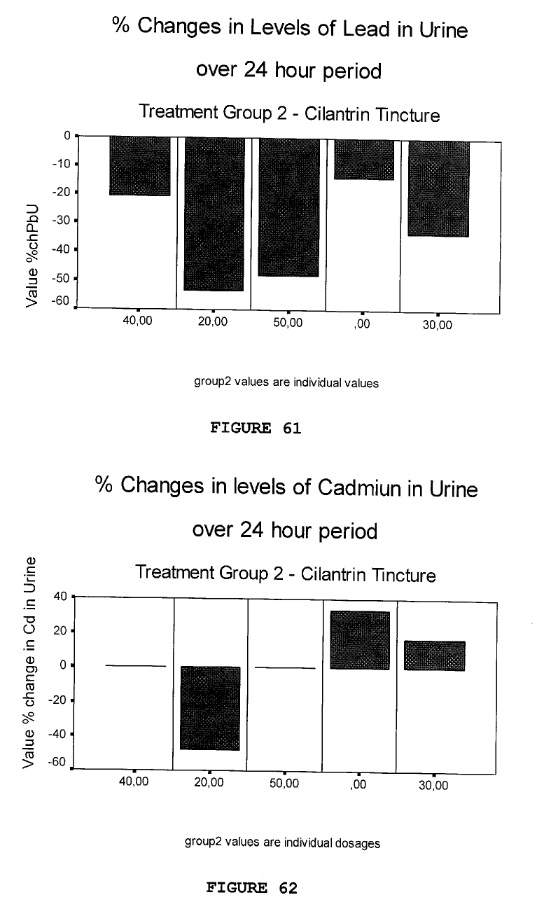

FIG. 61 shows percentage changes in levels of Lead in Urine over a 24 hour period in Treatment Group 2 using Cilantrin Tincture.

Cadmium in Urine—small increases noted but one is the placebo group so again this is coincidental.

FIG. 62 shows percentage changes in levels of Cadmium in Urine over a 24 hour period in Treatment Group 2 using Cilantrin Tincture.

Cadmium in Faeces—the pattern again is for % DECREASE for most dosages.

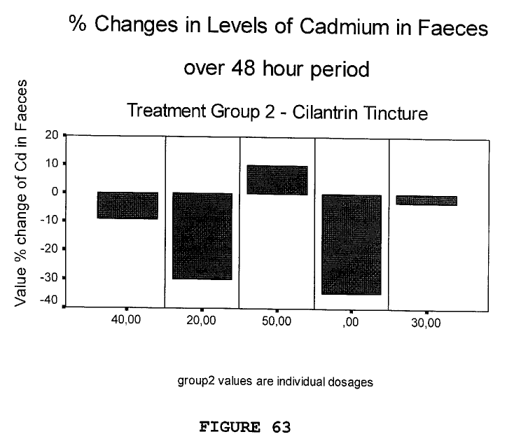

FIG. 63 shows percentage changes in levels of Cadmium in Faeces over a 48 hour period in Treatment Group 2 using Cilantrin Tincture.

### CONCLUSIONS

Cilantro Tincture (Coriander sativum)—there was also a consistent pattern in the Cilantro data, even though this was not a desired one as far as heavy metal detoxification is concerned. Cilantro appears to be a good intracellular detoxifier, but only if combined with other detoxifiers that can remove metals from the mesenchyme, before they have time to be reabsorbed back into the cell. The results for this protocol showed consistently that the body was retaining metals as opposed to eliminating them. This was clearly shown by a % DECREASE in the pre-post tests, as opposed to an increase.

1. A natural substance composition for the elimination of toxic metals from the body comprising a homeopathic detox factor, a nucleotide-peptide complex and coriander sativum.

2. A natural substance composition according to claim 1, in which the homeopathic detox factor contains cell wall decimated Chlorella, herb extracts and bacteriocin components of probiotics.

3. A natural substance composition according to claim 1, in which the nucleotide-peptide complex is Chlorella Growth Factor.

4. A natural substance composition according to claim 1, in which the composition contains between 10% and 50% Homeopathic Nano Detox Factor.

5. A natural substance composition according to claim 1, in which the composition contains 20% Homeopathic Nano Detox Factor.

6. A natural substance composition according to claim 1, in which the composition contains 30% Homeopathic Nano Detox Factor.

7. A natural substance composition according to claim 1, in which the composition contains 50% nucleotide-peptide complex.

8. A natural substance composition according to claim 1, in which the composition contains 40% nucleotide-peptide complex.

9. A natural substance composition according to claim 1, in which the composition contains between 20% and 40% of Cilantro.

10. A natural substance composition according to claim 1, in which the composition contains 30% of Cilantro.

11. A natural substance composition according to claim 2, in which the nucleotide-peptide complex is Chlorella Growth Factor.

12. A natural substance composition according to claim 11, in which the composition contains between 10% and 50% Homeopathic Nano Detox Factor.

13. A natural substance composition according to claim 12, in which the composition contains 50% nucleotide-peptide complex.

14. A natural substance composition according to claim 12, in which the composition contains 40% nucleotide-peptide complex.

15. A natural substance composition according to claim 12, in which the composition contains between 20% and 40% of Cilantro.

16. A natural substance composition according to claim 12, in which the composition contains 30% of Cilantro.

17. A natural substance composition according to claim 2, in which the composition contains 20% Homeopathic Nano Detox Factor.

18. A natural substance composition according to claim 17, in which the composition contains 50% nucleotide-peptide complex.

19. A natural substance composition according to claim 2, in which the composition contains 30% Homeopathic Nano Detox Factor.

20. A natural substance composition according to claim 19, in which the composition contains 50% nucleotide-peptide complex.
